(19) United States
(12) Patent Application Publication
(10) Pub. No.: US 2007/0298465 A1
(43) Pub. Date: Dec. 27, 2007

(54) COMPOSITIONS AND METHODS OF USE FOR VARIANT HELICASES

(75) Inventors: Timothy M. Lohman, Kirkwood, MO (US); George H. Gauss, Bozeman, MT (US); Wei Cheng, San Ramon, CA (US); Katherine Brendza, San Carlos, CA (US)

Correspondence Address: POLSINELLI SHALTON FLANIGAN SUELTHAUS PC, 700 W. 47TH STREET, SUITE 1000, KANSAS CITY, MO, 64112-1802, US

(73) Assignee: WASHINGTON UNIVERSITY IN ST. LOUIS, St. Louis, MO (US)

(21) Appl. No.: 11/558,552

(22) Filed: Nov. 10, 2006

Related U.S. Application Data
(60) Provisional application No. 60/735,549, filed on Nov. 10, 2005.

(51) Int. Cl. C12N 9/24; C12P 19/26

(52) U.S. Cl. 435/91.2; 435/195

(57) ABSTRACT

Generally speaking, the present invention relates to variant helicases that lack structural autoinhibition of helicase activity. In particular, the invention provides a composition and a kit comprising a helicase that lacks structural autoinhibition, and a method of unwinding a double helix comprising contacting the double helix with a helicase that lacks structural autoinhibition.

### CROSS REFERENCE TO RELATED APPLICATIONS

This application claims priority from U.S. Provisional Application Ser. No. 60/735,549 filed on Nov. 10, 2005, which is hereby incorporated by reference in its entirety.

### GOVERNMENTAL RIGHTS IN INVENTION

This work was supported by the U.S. Department of Health and Human Services/National Institutes of Health grant number R01GM45948. The U.S. Government has certain rights in this invention.

### FIELD OF THE INVENTION

Generally speaking, the invention relates to variant helicases that lack structural autoinhibition of helicase activity.

### BACKGROUND OF THE INVENTION

In vitro, nucleic acid double helixes may be separated into complementary single strands by heating the double helix till it denatures, or melts, into its complementary single strands. Such heat denaturation is typically used for in vitro assays that require a double helix to be separated into complementary single strands, such as PCR assays and in vitro replication of genomic DNA. However, these in vitro assays are limited because of their dependence on a high heat source, and by the fact that the high heat may damage the double helix.

Helicases are a ubiquitous class of enzymes that use the binding and hydrolysis of nucleoside triphosphates to catalyze the separation of a double helix into its complementary single strands in vivo. Helicases theoretically provide an alternative to heat denaturation. The use of helicases for in vitro assays, however, is limited because most helicases need to oligomerize before they possess helicase activity. Therefore, there is a need for a helicase that possesses helicase activity as a monomer.

### BRIEF SUMMARY OF THE INVENTION

Among the several aspects of the invention is provided an in vitro method of unwinding a double helix. The method comprises contacting a double helix with a variant helicase that lacks structural autoinhibition of helicase activity.

Another aspect of the invention provides an in vitro composition. The composition comprises a variant helicase that lacks structural autoinhibition of helicase activity and a polymerase.

An additional aspect of the invention provides a kit. The kit comprises a variant helicase that lacks structural autoinhibition of helicase activity and a polymerase.

### FIGURE LEGENDS

FIG. 1 depicts diagrams showing the construction of the RepΔ2B mutant and a western blot of the purified RepΔ2B proteins. (A) Ribbon diagrams of the wtRep crystal structure in the “open” conformation (Korolev et al. 1997 Cell 90:635-47) and the hypothetical structure of RepΔ2B. The four subdomains within wtRep are 1A (yellow), 1B (green), 2A (red), and 2B (blue). In RepΔ2B, amino acids Thr-375 to Arg-542, constituting the 2B subdomain, were deleted and replaced by three Gly residues (shown in blue). For the hexahistidine-tagged RepΔ2B, the location of the N-terminal histidine tag is marked in black in the hypothetical RepΔ2B structure. The organization of the subdomains within the primary structure is shown below each structure. (B) SDS-polyacrylamide (12%) gel of the purified wtRep and RepΔ2B proteins (1 μg each).

FIG. 2 depicts a comparison of the sizes of the 2B domains within a representative number of SF1 and SF2 superfamily helicases. Sequence comparisons show the number of amino acids (red number) inserted between helicase motif IV and motif V. The numbers in parenthesis represent the amino acid residue numbers of the first residue shown in motif IV and the last residue shown in motif V. The numbers in red are the numbers of the residues between these two motifs. The names of the SF1 helicases are indicated in black, and the names of the SF2 helicases are indicated in blue. (SWISS-PROT sequence ID: Rep, P09980; PcrA, P56255; UvrD, P03018; RecB, P08394; RecD, P04993; PIF1, P07271; HeID, P15038; Srs2, P12954; Dda, P32270; UL5, P10189; NS3, P27958; eIF4A, P10081; UvrB, Q56243; PriA, P17888; recQ, P15043; Sgs1, P35187; BLM, P54132; WRN, Q14191; recG, P24230).

FIG. 3 depicts ribbon diagrams showing the superposition of the two conformations (“open” and “closed”) of the wtRep protein in complex with ssDNA and ADP. Six nucleotides are shown in purple, and the ADP is shown in orange [modified from Korolev et al., (1997) Cell 90:635-47]. The two conformations differ in the orientation of the 2B domain, shown in light blue for the open form and deep blue for the closed form. The hinge region connecting the 2B domain to the 2A domain, and about which the 2B domain rotates by ≈130° to convert from one form to the other, is shown in yellow for the open form and red for the closed form.

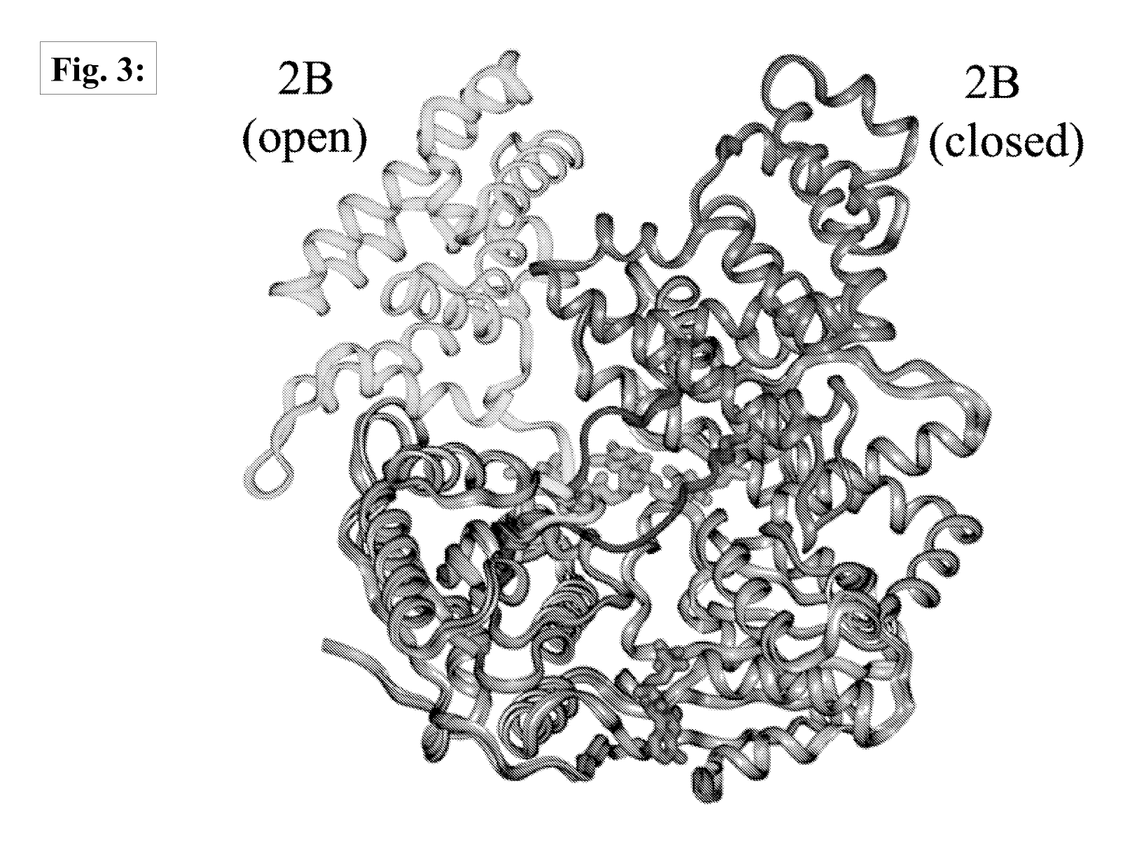

FIG. 4 depicts graphs showing the STO kinetic studies of Rep-catalyzed DNA unwinding of an 18-bp DNA substrate with a 3′-(dT)₂₀ tail examined using rapid chemical quenched-flow methods. (A) Comparisons of the fraction of DNA molecules unwound as a function of time for wtRep (◯), +HRepΔ2B (▪), and RepΔ2B (□). All time courses were performed in Buffer U at 25° C., with preincubation concentrations of 30 nM (protein) and 2 nM (DNA). The time courses were fit individually to Eq. 1 (see Examples), constraining the number of steps n=4, to obtain estimates of k_{obs} and k_{NP}, and the extent of unwinding, A_{T}. The solid lines are simulations using Eq. 1 (see Examples) and the best fit parameters determined from nonlinear least squares analysis. (B) The total unwinding amplitudes, A_{T}, as a function of protein concentration for wtRep (◯), +HRepΔ2B (▪), and RepΔ2B (□).

FIG. 5 depicts a graph showing the single-turnover DNA unwinding rates, k_{obs}, for unwinding of an 18-bp DNA substrate with a 3′-(dT)₂₀ tail examined using rapid chemical quenched-flow methods. The observed unwinding rates, k_{obs}, are plotted as a function of the preincubation protein concentration for wtRep (open circles), +HRepΔ2B (filled squares), and RepΔ2B (open squares). All experiments were performed with a preincubation (DNA) of 2 nM in Buffer U, 25° C. The time courses were fit individually to Eq. 1 (see Examples), constraining the number of steps, n=4, to obtain estimates of k_{obs} and k_{NP}, and the extent of unwinding, A_{T}.

FIG. 6 depicts a graph showing the time courses for E. coli cells expressing wt Rep vs. RepΔ2B. The cell density of each culture was assumed to be proportional to the optical density at 600 nm (OD₆₀₀), which is plotted on a logarithmic scale vs. time. Filled diamonds, E. coli CK11 D rep/plWcl; open circles, CK11D rep/plWcl carrying pRepO (expressing wtRep); open squares, CK11D rep/plWcl carrying pRepOΔ2B (expressing RepΔ2B).

FIG. 7 depicts graphs showing the sedimentation equilibrium ultracentrifugation of wtRep and RepΔ2B proteins. Experiments were performed at rotor speeds of 23,000 (red), 28,000 (blue), and 34,000 (green) rpm in Buffer M, 200 mM NaCl at 25° C., monitoring absorbance at 230 nm. (A) Data for RepΔ2B (2 μM loading concentration). Solid lines are simulations using Eq. 2 (see Examples) and the best-fit parameters obtained from a global NLLS fit of all data to a single ideal species (n=1 in Eq. 2, see Examples). A plot of the residuals for each data set is shown below. Note that the residuals are all centered around zero but shifted along y axis for clarity. (B) Data for wtRep (1.5 μM loading concentration). Solid lines are simulations using Eq. 2 (see Examples) and the best-fit parameters obtained from a global NLLS fit of all data to a single ideal species (n=1 in Eq. 2, see Examples). A plot of the residuals for each data set is shown below. Note that the residuals are all centered around zero but shifted along y axis for clarity.

FIG. 8 depicts a schematic of DNA substrates. All substrates possess a (dT20) tail. DNA II and III represent a series of substrates with varying duplex length, L. The top strand in DNA II is labeled on its 5′ end with ³²P. DNA sequences of the substrates used are given in Table 1.

FIG. 9 depicts graphs demonstrating that the RepΔ2Bmonomer is an active helicase. (A) Sedimentation equilibrium DNA concentration profiles (monitoring Cy3 absorbance of DNA I) at 18,000 (red), 22,000 (blue), and 27,000 (green) rpm. The solid curves are simulations based on global NLLS fits of the data to Eq. 2 (see Examples), with residual plots below. (B) STO kinetics of unwinding of DNA III catalyzed by RepΔ2B [increase in Cy3 fluorescence (red) and decrease in Cy5 fluorescence (blue)]; no DNA unwinding (no Cy3 fluorescence increase) was catalyzed by wtRep monomer (dark green).

FIG. 10 depicts a graph of the sedimentation equilibrium of RepΔ2B bound to DNA I. The values of the apparent molecular mass, M, of the RepΔ2B-DNA complex (where PD is a protein monomer bound to DNA and P2D is two protein monomers bound to DNA), obtained from NLLS analysis, as a function of the RepΔ2B/DNA molar ratio of the loading concentrations.

FIG. 11 depicts graphs showing the sedimentation equilibrium ultracentrifugation of wtRep in the presence of an excess of DNA I, which contains an 18-bp duplex and 3′-(dT₂₀) tail with Cy3 covalently attached at the 5′ end of the bottom strand (see cartoon in Inset). Experiments were performed at rotor speeds of 22,000 (red), 27,000 (blue), and 32,000 (green) rpm in Buffer M (plus 200 mM NaCl) at 25° C. with loading concentrations of 1.5 μM wtRep and 3 μM DNA, monitoring absorbance of the Cy3 labeled DNA at 550 nm. (A) Experimental data (open circles) and simulations (solid lines) based on Eq. 2 (see Examples) and the best-fit parameters obtained from a global NLLS fit of all data to a two-component model (n=2 in Eq. 2, see Examples), with the two components representing free DNA and DNA bound by one wtRep monomer. A plot of the residuals for each data set is shown below the data. Note that the residuals are all centered around zero but shifted along y axis for clarity. (B) The values of M for the wtRep-DNA complex obtained from NLLS analysis as a function of the ratio of the loading concentrations.

FIG. 12 depicts graphs showing the sedimentation equilibrium ultracentrifugation of RepΔ2B in the presence of an excess of DNA I, which contains an 18-bp duplex with a 3′-(dT₂₀) tail with Cy3 covalently attached at the 5′ end of the bottom strand (see cartoon in Inset). Experiments were performed at rotor speeds of 18,000 (blue), 22,000 (red), 27,000 (green), and 33,000 (gray) rpm in Buffer M plus 50 mM NaCl at 25° C. with loading concentrations of 2.0 μM RepΔ2B and 4 μM DNA, monitoring absorbance of the Cy3 labeled DNA at 550 nm. (A) Experimental data (open circles) and simulations (solid lines) based on Eq. 2 (see Examples) and the best-fit parameters obtained from a global NLLS fit of all data to a two-component model (n=2 in Eq. 2, see Examples), with the two components representing free DNA and DNA bound by one RepΔ2B monomer. A plot of the residuals is shown below the data. Note that the residuals are all centered around zero but shifted along the y axis for clarity. (B) The values of M for the RepΔ2B-DNA complex obtained from NLLS analysis as a function of the ratio of the loading concentrations.

FIG. 13 depicts graphs demonstrating ssDNA translocation by wtRep and RepΔ2B monomers. Stopped-flow kinetics experiments were performed as described (Fischer et al. 2004 J. Mol. Biol. 344:1287-1309) with a series of 5′-Cy3-(dTL) (L=54, 64, 74, 84, 94, 104, 114, and 124 nt), monitoring Cy3 fluorescence with wtRep (A), and RepΔ2B (B). For clarity, only the time courses for L=64, 84,104, and 124 nt and control experiments with 5′-(dT79)-Cy3 (black), and 5′-(dT59)-Cy3 (orange) are shown. Solid lines are simulations using Eq. 3 (RepΔ2B) or Eq. 4 (wtRep) and the kinetic parameters from a global NLLS fit of time courses obtained with all DNA lengths (see Examples).

FIG. 14 depicts a graph showing the dependence of DNA unwinding amplitude on wtRep protein concentration in Buffer M plus 50 mM NaCl. STO DNA unwinding kinetics experiments were performed in the quenched-flow in Buffer M (plus 50 mM NaCl, 2.1 mM MgCl₂, and 0.1 mg/ml BSA at 25° C.) using 40 nM DNA II and varying wtRep concentrations (40-800 nM). Experiments were initiated by the addition of ATP to a final concentration of 1.5 mM. The ATP solution also included an excess of protein trap to ensure that only a single cycle of DNA unwinding occurred.

FIG. 15 depicts graphs demonstrating STO DNA unwinding kinetics catalyzed by the RepΔ2B monomer. (A) DNA unwinding monitored by the increase in Cy3 fluorescence at several [NaCl]. Identical experiments performed with wtRep monomer (orange). (B and C) DNA unwinding dependence on duplex length. STO quenched-flow experiments at 200 mM NaCl (B). STO stopped-flow experiments at 50 mM NaCl (C). Solid lines are simulations based on Eq. 1 (see Examples) and the kinetic parameters from a global NLLS fit of the unwinding time courses (FIG. 16).

FIG. 16 depicts a table showing the kinetic parameters for unwinding of DNA III determined from NLLS analysis of the data in FIG. 4A using Scheme 1 (Eq. 5, see Examples), assuming a step size m=L/n=4 bp. Conditions: Buffer M at 25° C. plus the indicated [NaCl]. *Fraction of DNA bound by protein in complexes productive for DNA unwinding. †total amplitude of unwinding (arbitrary fluorescence units).

### DETAILED DESCRIPTION OF THE INVENTION

Most helicases do not possess helicase activity until they oligomerize. The oligomerization requirement overcomes a structural inhibition inherent in the helicase monomer that prohibits the monomer from possessing helicase activity. The present invention encompasses a variant helicase that lacks structural autoinhibition, and therefore, possesses helicase activity as a monomer. Advantageously, a monomeric helicase that possesses helicase activity is capable of unwinding a nucleic acid double helix at temperatures lower than that required to denature the double helix. This eliminates the risk of exposing nucleic acid samples to high temperatures, and it eliminates the need for a source of heat in research applications that require denaturation of the double helix, such as DNA replication, genomic DNA replication, PCR, in vitro translation, in vitro transcription, and reverse transcription.

The Variant Helicase

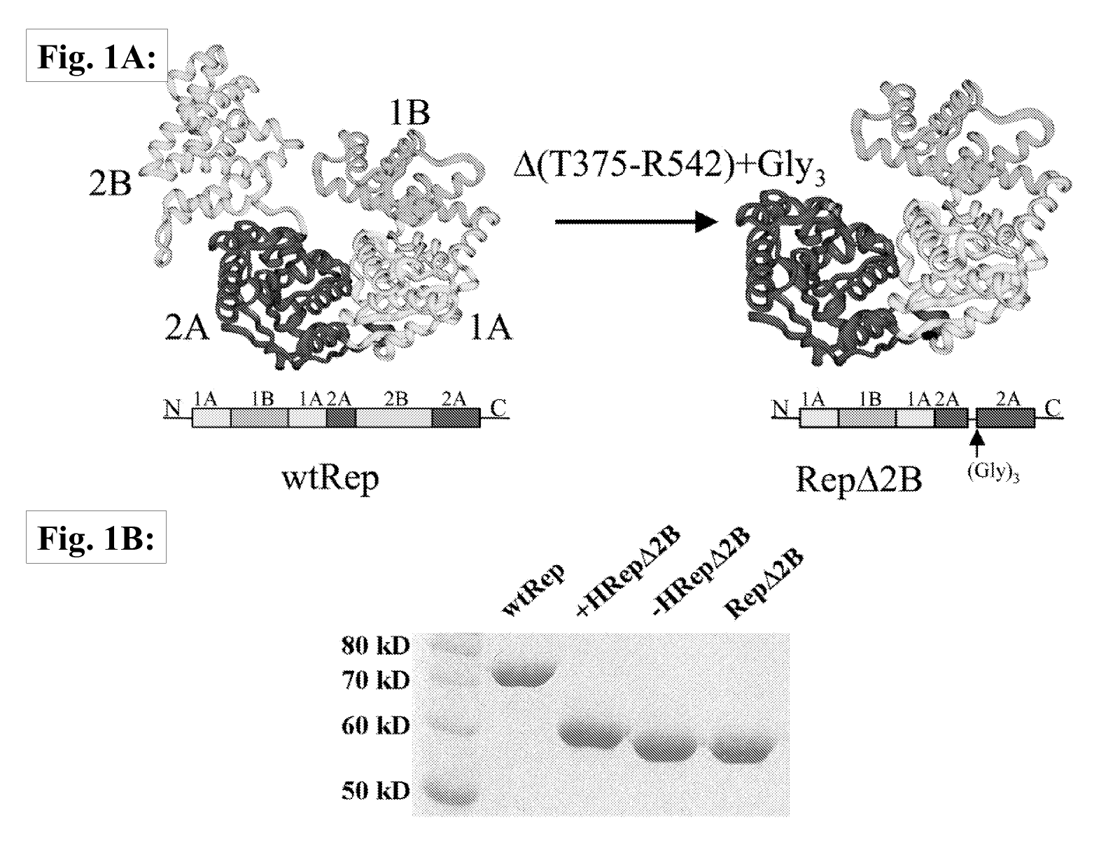

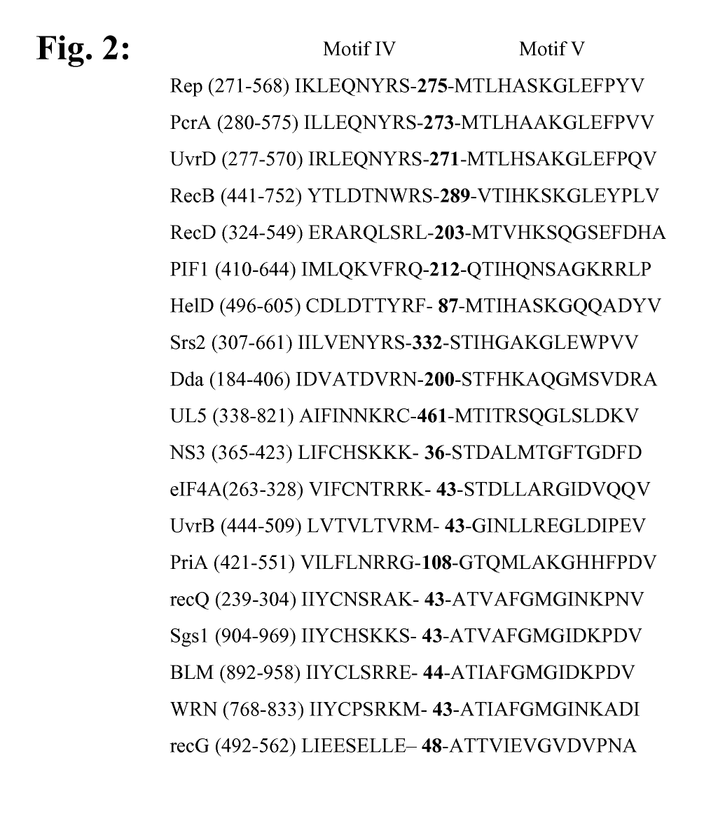

In one embodiment, a variant helicase of the invention that lacks structural autoinhibition is derived from an SF1 or SF2 helicase. SF1 and SF2 helicase family members share a common protein structure comprising seven highly conserved helicase motifs that define the helicase superfamilies (Gorbalenya and Koonin, 1993 Curr. Opin. Stuct. Biol. 3:419-29). The typical SF1 or SF2 helicase monomer is comprised of four separate domains (1A, 1B, 2A, and 2B) based on tertiary protein structure (FIG. 1). The seven highly conserved helicase motifs important for helicase activity are generally located in two of the four domains, 1A and 2A. The 2B subdomain is an insert between helicase motifs IV and V in the 2A domain (FIG. 1). The size and amino acid sequences of the 2B domain varies within the SF1 and SF2 families (FIG. 2). However, the common location of the 2B domain between families allows the invention to utilize helicases from both families. In one embodiment, a variant helicase is derived from an SF1 or an SF2 helicase. In another embodiment, a variant helicase is derived from an SF1 helicase selected from the group comprising Rep, PcrA, UvrD, RecB, RecD, PIF1, HeID, Srs2, Dda, and UL5. In yet another embodiment, a variant helicase is derived from an SF2 helicase selected from the group comprising NS3, eIF4A, UvrB, PriA, recQ, Sgs1, BLM, WRN, and recG. In a further embodiment of the invention, a variant helicase is derived from UvrD. In yet another embodiment, a variant helicase is derived from Rep. In still yet another embodiment, a variant helicase possesses 3′ to 5′ activity. Alternatively, a variant helicase possesses 5′ to 3′ activity. In an exemplary embodiment, a variant helicase can initiate unwinding from the ends of a blunt-ended double helix. In all embodiments described above, the variant helicase lacks structural autoinhibition and therefore possesses helicase activity as a monomer.

In another embodiment of the invention, the 2B domain of a variant helicase does not structurally inhibit helicase activity. In one embodiment, the 2B domain of an SF1 or SF2 helicase does not structurally inhibit helicase activity. In yet another embodiment, the 2B domain of an SF1 helicase does not structurally inhibit helicase activity. In another embodiment, the 2B domain of UvrD does not structurally inhibit helicase activity. In another embodiment, the 2B domain of Rep does not structurally inhibit helicase activity. In one embodiment, a portion of the 2B domain is mutated to create a variant helicase that lacks structural autoinhibition. In another embodiment, a substantial portion of the 2B domain is deleted to create a variant helicase. In yet another embodiment, the 2B domain is deleted to create a variant helicase. In yet another embodiment, a portion, or a substantial portion, of the 2B domain of UvrD is mutated to create a variant helicase. In still yet another embodiment, the 2B domain of UvrD is deleted to create a variant helicase. Alternatively, a portion, or a substantial portion, of the 2B domain of Rep is mutated to create a variant helicase. In another embodiment, the 2B domain of Rep is deleted to create a variant helicase. In yet another embodiment, amino acids THR-375 to ARG-542 of the Rep helicase are removed and replaced with a flexible linker to create a variant helicase. In an additional embodiment, amino acids THR-375 to ARG-542 of the Rep helicase are removed and replaced with one to ten amino acids to create a variant helicase. Alternatively, amino acids THR-375 to ARG-542 of the Rep helicase are removed and replaced with one to five amino acids to create a variant helicase. In still another alternative, amino acids THR-375 to ARG-542 of the Rep helicase are removed and replaced with three glycines to create a variant helicase. In all embodiments described above, the variant helicase lacks structural autoinhibition and therefore possesses helicase activity as a monomer.

In an exemplary embodiment, a variant helicase is comprised of the amino acid sequence SEQ ID NO:1. In an additional embodiment, a variant helicase is comprised of an amino acid sequence that is at least 50% identical to SEQ ID NO:1. More typically, a variant helicase is comprised of an amino acid sequence that is at least 75% identical to the amino acid sequence of SEQ ID NO:1, and even more typically, 90% identical to the amino acid sequence of SEQ ID NO:1. In a further embodiment, a variant helicase is comprised of an amino acid sequence that is at least 95% identical to the amino acid sequence of SEQ ID NO:1, and even further, 99% identical to the amino acid sequence of SEQ ID NO:1. In each of these embodiments, the amino acid sequence encodes a variant helicase that lacks structural autoinhibition of helicase activity and therefore possesses helicase activity as a monomer.

In a second exemplary embodiment, a variant helicase is comprised of the nucleic acid sequence SEQ ID NO:2. In an additional embodiment, a variant helicase is comprised of a nucleic acid sequence that is at least 50% identical to SEQ ID NO:2. More typically, a variant helicase is comprised of a nucleic acid sequence that is at least 75% identical to the sequence of SEQ ID NO:2, and even more typically, 90% identical to the nucleic acid sequence of SEQ ID NO:2. In a further embodiment, a variant helicase is comprised of a nucleic acid sequence that is at least 95% identical to the nucleic acid sequence of SEQ ID NO:2, and even further, 99% identical to the nucleic acid sequence of SEQ ID NO:2. In each of these embodiments, the nucleic acid sequence encodes a variant helicase that lacks structural autoinhibition of helicase activity and therefore possesses helicase activity as a monomer.

In determining whether a polypeptide or a nucleic acid is substantially homologous or shares a certain percentage of sequence identity with a sequence of the invention, sequence similarity may be determined by conventional algorithms, which typically allow introduction of a small number of gaps in order to achieve the best fit. In particular, “percent identity” of two polypeptides or two nucleic acid sequences is determined using the algorithm of Karlin and Altschul (Proc. Natl. Acad. Sci. USA 87:2264-2268, 1993). Such an algorithm is incorporated into the NBLAST and XBLAST programs of Altschul et al. (J. Mol. Biol. 215:403-410, 1990). BLAST nucleotide searches may be performed with the NBLAST program to obtain nucleotide sequences homologous to a nucleic acid molecule of the invention. Equally, BLAST protein searches may be performed with the XBLAST program to obtain amino acid sequences that are homologous to a polypeptide of the invention. To obtain gapped alignments for comparison purposes, Gapped BLAST is utilized as described in Altschul et al. (Nucleic Acids Res. 25:3389-3402, 1997). When utilizing BLAST and Gapped BLAST programs, the default parameters of the respective programs (e.g., XBLAST and NBLAST) are employed. See http://www.ncbi.nlm.nih.gov for more details.

The invention also encompasses the use of nucleotide sequences other than a sequence that encodes a polypeptide having an amino acid sequence of SEQ ID NO:1 or an amino acid sequence of SEQ ID NO:2. Typically, these nucleotide sequences will hybridize under stringent hybridization conditions (as defined herein) to all or a portion of the nucleotide sequences described above or their complement. The hybridizing portion of the hybridizing nucleic acids is usually at least 15 (e.g., 20, 25, 30, or 50) nucleotides in length. The hybridizing portion of the hybridizing nucleic acid is at least 80%, preferably, at least 90%, and is more preferably, at least 95% identical to the sequence of a portion or all of a nucleic acid sequence encoding a variant helicase suitable for use in the present invention, or its complement.

Hybridization of the oligonucleotide probe to a nucleic acid sample is typically performed under stringent conditions. Nucleic acid duplex or hybrid stability is expressed as the melting temperature or Tₘ, which is the temperature at which a probe dissociates from a target DNA. This melting temperature is used to define the required stringency conditions. If sequences are to be identified that are related and substantially identical to the probe, rather than identical, then it is useful to first establish the lowest temperature at which only homologous hybridization occurs with a particular concentration of salt (e.g., SSC or SSPE). Then, assuming a 1% mismatching results in a 1° C. decrease in the Tₘ, the temperature of the final wash in the hybridization reaction is reduced accordingly. For example, if sequences have greater than 95% identity with the probe is sought, the final temperature is approximately decreased by 5° C. In practice, the change in Tₘ can be between 0.5 and 1.5° C. per 1% mismatch. Stringent conditions involve hybridizing at 68° C. in 5×SSC/5×Denhardt's solution/1.0% SDS, and washing in 0.2×SSC/0.1% SDS at room temperature. Moderately stringent conditions include washing in 3×SSC at 42° C. The parameters of salt concentration and temperature can be varied to achieve the optimal level of identity between the probe and SEQ ID NO:1 or SEQ ID NO:2. Additional guidance regarding such conditions is readily available in the art, for example, by Sambrook et al., 1989, Molecular Cloning, A Laboratory Manual, Cold Spring Harbor Press, N.Y.; and Ausubel et al., (eds.), 1995, Current Protocols in Molecular Biology, (John Wiley & Sons, N.Y.) at Unit 2.10.

A number of methods may be employed to determine whether a variant helicase has substantially similar helicase activity relative to a variant helicase of the invention. In particular, the subject variant helicase, if suitable for use in the invention, will possess helicase activity as a monomer. In order to determine whether a particular variant helicase can function in this manner, the procedures detailed in the examples may be followed.

A variant helicase of the invention may be synthesized, produced by recombinant technology, or purified from cells. In one embodiment, a variant helicase of the present invention may be expressed in cell and cell-free systems (e.g. Jermutus L, et al., Curr Opin Biotechnol. October 1998; 9(5):534-48) from encoding polynucleotides, such as described below, or polynucleotides optimized for selected expression systems made by back-translating the subject polypeptides according to computer algorithms (e.g. Holler et al. (1993) Gene 136, 323-328; Martin et al. (1995) Gene 154, 150-166). In other embodiments, any of the molecular and biochemical methods known in the art are available for biochemical synthesis, molecular expression and purification of a variant helicase, see e.g. Molecular Cloning, A Laboratory Manual (Sambrook, et al. Cold Spring Harbor Laboratory), Current Protocols in Molecular Biology (Eds. Ausubel, et al., Greene Publ. Assoc., Wiley-Interscience, New York).

The various nucleic acid sequences mentioned above can be obtained using a variety of different techniques known in the art. The nucleotide sequences, as well as homologous sequences encoding a suitable variant helicase, can be isolated using standard techniques, or can be purchased or obtained from a depository. Once the nucleotide sequence is obtained, it can be amplified for use in a variety of applications, as further described below.

The invention also encompasses production of nucleotide sequences that encode suitable variant helicases, derivatives, or fragments thereof, that may be made by any method known in the art, including by synthetic chemistry. After production, the synthetic sequence may be inserted into any of the many available expression vectors and cell systems using reagents well known in the art. Moreover, synthetic chemistry may be used to introduce additional mutations into a nucleotide sequence encoding a suitable variant helicase.

Method of Unwinding DNA

Another aspect of the present invention encompasses a method for unwinding a nucleic acid double helix with a variant helicase of the invention. The nucleic acid double helix may be selected from the group comprising genomic DNA, a PCR template, a template for in vitro transcription, a template for in vitro translation, RNA, and a template for reverse transcription.

In general, the method of unwinding the nucleic acid double helix is comprised of contacting the nucleic acid double helix with a variant helicase under temperature and buffer conditions (the “unwinding buffer”) suitable for unwinding of the helix. In one embodiment, a variant helicase unwinds the helix at a temperature between about 20° C. and 70° C. In another embodiment, a variant helicase unwinds the helix at a temperature between about 20° C. and 50° C. In yet another embodiment, a variant helicase unwinds the helix at a temperature between 30° C. and 70° C. In still yet another embodiment, a variant helicase unwinds the helix at room temperature.

In a further embodiment, the unwinding buffer is comprised of a salt and a reducing agent. In another embodiment, the unwinding buffer is comprised of a chloride salt and a reducing agent. In another embodiment, the unwinding buffer is comprised of a Tris salt, a chloride salt, and a reducing agent. The chloride salt may be selected from the group comprising Tris HCl, NaCl, MgCl₂, and KCl. The Tris salt can be Tris HCl or TrisAcetate. Examples of reducing agents include 2-mercaptoethanol, dithiothreitol, Tris[2-carboxyethyl]phosphine, and 2-Mercaptoethylamine HCl. In one embodiment, the unwinding buffer is comprised of Tris HCl, NaCl, MgCl₂, 2-mercaptoethanol, glycerol, and bovine serum albumin (BSA). Alternatively, the unwinding buffer is comprised of between about 10 and 30 mM Tris HCl, between about 3 and 10 mM NaCl, between about 0.5 and 2.5 mM MgCl₂, between about 0 and 10 mM 2-mercaptoethanol, between about 2 to 20% (vol/vol) glycerol, and between about 0 to 0.5 mg/ml BSA. In another alternative, the unwinding buffer is comprised of between about 15 and 25 mM Tris HCl, between about 4 and 8 mM NaCl, between about 1.0 and 2.0 mM MgCl₂, between about 2 and 8 mM 2-mercaptoethanol, between about 8 to 15% (vol/vol) glycerol, and between about 0 to 0.25 mg/ml BSA. In still another alternative, the unwinding buffer is comprised of about 20 mM Tris HCl, about 6 mM NaCl, about 1.7 mM MgCl₂, about 5 mM 2-mercaptoethanol, about 10% (vol/vol) glycerol, and about 0.1 mg/ml BSA. In a certain embodiment, the unwinding buffer is titrated to a pH ranging from about 6.8 to 8.2. Alternatively, the unwinding buffer is titrated to a pH ranging from about 7.2 to 7.8. In another alternative, the unwinding buffer is titrated to a pH of 7.5.

In another embodiment, the unwinding buffer is further comprised of ATP and a mock DNA substrate. In one alternative, the unwinding buffer is further comprised of between about 1 mM to 5 mM ATP and between about 1 μM to 14 μM mock DNA substrate. In another alternative, the unwinding buffer is further comprised of between about 2 mM to 4 mM ATP and between about 5 μM to 10 μm mock DNA substrate. In yet another alternative, the unwinding buffer is further comprised of about 3 mM ATP and about 8 μM mock DNA substrate.

Helix unwinding encompasses both complete separation of the two strands of the nucleic acid double helix and partial unwinding of the two strands of the double helix. Partial unwinding encompasses one or more rounds of unwinding as described in the examples. Both complete and partial unwinding can be measured using methods known in the art. An example of a method used to detect double helix unwinding is the single turnover (STO) assay, further described in the examples.

In an embodiment of the invention, the method utilizes a variant helicase in an isothermal PCR assay. In general, isothermal PCR refers to a PCR that does not require thermocycling. A protocol for isothermal PCR has been described (Vincent et al., EMBO Reports 5:795-800 (2004); An et al., J. Biol. Chem. 280:28952-58 (2005); both herein incorporated by reference). Briefly, a nucleic acid double helix is contacted with a helicase under conditions such that the two strands of the helix are separated. Sequence specific primers anneal to the single-stranded nucleic acid, and polymerases extend the primers to produce two progeny double-helices. The process is then repeated to amplify the target sequence to the desired concentration. In a particular embodiment, the isothermal PCR is performed at a temperature between about 20° C. and 70° C. In another embodiment, the isothermal PCR is performed at a temperature between about 20° C. and 50° C. In yet another embodiment, the isothermal PCR is performed at a temperature between 30° C. and 70° C. In an exemplary embodiment, the isothermal PCR is performed at room temperature. In another particular embodiment, the isothermal PCR buffer is comprised of unwinding buffer, dNTPs, and primers. In a further embodiment, the primers are labeled. Examples of appropriate primer labels are fluorophores, biotin-strepavidin, or radioactive labels. The dNTPs can be selected from the group comprising dATP, dGTP, dCTP, dTTP, or dUTP.

In another embodiment of the invention, the method utilizes a variant helicase to replicate genomic DNA. Replicating genomic DNA using high temperatures to melt the double helix is difficult because the high temperatures tend to damage the long genomic DNA. Therefore, one embodiment of the present invention is a method to replicate genomic DNA at room temperature. The protocol for replicating genomic DNA is similar to the one described above for isothermal PCR. Previously, attempts to replicate genomic DNA without high temperatures have hinged on chemically denaturing the double helix. The present invention allows unwinding of the genomic double-helix such that denaturing of the double helix, whether by high temperatures or by chemicals, is not necessary.

In yet another embodiment of the invention, the method utilizes a variant helicase to transcribe RNA. Methods to transcribe RNA are known by those skilled in the art. In general, the transcription template is contacted with an RNA polymerase under conditions that allow transcription. Typically, the DNA template is initially denatured. The present invention encompasses a transcription reaction that does not require helix denaturation, but instead utilizes a variant helicase to unwind the DNA duplex. In one embodiment, the transcription is performed at room temperature. In another embodiment, the method is further comprised of translating the transcribed product.

In still another embodiment of the invention, the method utilizes a variant helicase in reverse transcriptase PCR (RT-PCR). Typically, the RNA template is exposed to reverse transcriptase under conditions that allow the RNA template to be reverse transcribed to DNA. The reverse transcribed DNA is then used as a template for an isothermal PCR reaction, as described previously.

Composition of a Variant Helicase and a Polymerase

A further aspect of the present invention is a composition of a variant helicase of the invention and a polymerase. The polymerase can be selected from the group comprising DNA polymerases, RNA polymerases, viral polymerases, and thermostable polymerases. Examples of DNA polymerases include Pol I, Pol II, Pol III, Pol α, Pol β, Pol γ, Pol ε, Pol ζ, and Pol δ. Examples of RNA polymerases include RNAP, RNA polymerase I, RNA polymerase II, and RNA polymerase III. Examples of viral polymerases include DNA-dependent RNA polymerases, RNA-dependent DNA polymerases, and RNA-dependent RNA polymerases. Examples of thermostable polymerases include Taq polymerase, Bst polymerase, Tbr polymerase, Tfl polymerase, Tgo polymerase, and Tth polymerase. In one embodiment, the composition is comprised of a variant helicase and a Pol I family polymerase. Examples of Pol I family polymerases include the Klenow fragments of Escherichia coli and Bacillus DNA polymerase I, Thermus aquaticus DNA polymerase, and the T7 RNA and DNA polymerases.

In another embodiment, the composition of a variant helicase and a polymerase is further comprised of a nucleic acid double helix. The double helix can be selected from the group comprising genomic DNA, a PCR template, a template for in vitro transcription, a template for in vitro translation, double-stranded RNA, and a template for reverse transcriptase. In one exemplary embodiment, the double helix is genomic DNA. In another exemplary embodiment, the double helix is a PCR template.

In yet another embodiment, the composition is further comprised of dNTPs, primers, and unwinding buffer. The dNTPs can be selected from the group comprising dATP, dGTP, dCTP, dTTP, or dUTP. The primers can be random primers, or sequence specific primers. In a particular embodiment, the primers are labeled. Examples of appropriate primer labels are fluorophores, biotin-strepavidin, or radioactive labels.

In an exemplary embodiment, the composition is comprised of a variant helicase, a polymerase, a PCR template, dNTPs, sequence specific primers, and buffer. In a second exemplary embodiment, the composition is comprised of a variant helicase, a polymerase, genomic DNA, random primers, dNTPs, and buffer.

Kit Comprising the Composition of a Variant Helicase and a Polymerase

An additional aspect of the present invention is a kit comprised of a variant helicase of the invention and a polymerase. The polymerase can be selected from the group comprising DNA polymerases, RNA polymerases, viral polymerases, and thermostable polymerases. Examples of DNA polymerases include Pol I, Pol II, Pol III, Pol α, Pol β, Pol γ, Pol ε, Pol ζ, and Pol δ. Examples of RNA polymerases include RNAP, RNA polymerase I, RNA polymerase II, and RNA polymerase III. Examples of viral polymerases include DNA-dependent RNA polymerases, RNA-dependent DNA polymerases, and RNA-dependent RNA polymerases. Examples of thermostable polymerases include Taq polymerase, Bst polymerase, Tbr polymerase, Tfl polymerase, Tgo polymerase, and Tth polymerase. In one embodiment, the composition is comprised of a variant helicase and a Pol I family polymerase. Examples of Pol I family polymerases include the Klenow fragments of Escherichia coli and Bacillus DNA polymerase I, Thermus aquaticus DNA polymerase, and the T7 RNA and DNA polymerases.

In another embodiment, the kit is directed towards the replication of genomic DNA. In an exemplary embodiment, the kit is directed towards the isothermal replication of genomic DNA. In another embodiment, the kit is directed towards performing PCR. In a second exemplary embodiment, the kit is directed towards performing isothermal PCR. In yet another embodiment, the kit is directed towards in vitro transcription. In still another embodiment, the kit is directed towards in vitro translation. In a further embodiment, the kit is directed towards reverse transcription. In each of the above embodiments, the procedure of the kit is performed at room temperature.

Definitions

As used herein, “autoinhibition” means inhibition of helicase activity of a helicase monomer by a domain of the monomer. If a monomer lacks autoinhibition, then it possesses helicase activity as a monomer.

As used herein, “enzyme” means a protein, portion of a protein, or polypeptide that acts as a biochemical catalyst.

As used herein, “helicase activity” refers to the ability to enzymatically separate the strands of a double-helix.

As used herein, “mock DNA substrate” means an unlabelled substrate meant to serve as a trap for excess protein.

As used herein, “monomer” means a single protein or polypeptide. A monomer may be capable of binding to other proteins, including another copy of itself.

As used herein, “mutate” encompasses both changes to a nucleic acid or amino acid sequence as well as elimination or addition of a sequence or part of a sequence. Additionally, a mutated nucleic acid sequence may be expressed as a polypeptide. Methods to mutate a nucleic acid or amino acid sequence are commonly known in the art.

As used herein, “oligomerize” means for two or more proteins to associate.

As used herein, “PCR template” means a segment of DNA to be amplified using the PCR process. A PCR template can be between about 15 bp to 4 kb.

As used herein, “replicate” means contacting a nucleic acid with a polymerase such that a new strand of nucleic acid complementary to the original strand is produced.

As used herein, “room temperature” means the temperature range in which the reaction in question will proceed. The upper temperature of the range is defined by the denaturization temperature of the enzyme in question. The lower temperature of the range is the temperature below which the enzyme in question has no measurable activity.

As used herein, “structural autoinhibition” refers to a regulatory mechanism mediated by the tertiary and quaternary structure of a protein.

As used herein, “unwinding” means physically separating the two strands of a double helix using an enzyme. Unwinding encompasses both complete separation of the two strands of the nucleic acid double helix and partial unwinding of the two strands of the double helix. Partial unwinding encompasses one or more rounds of unwinding as described in the examples. As used herein, “melting” or “denaturation” means breaking the bonds between the two strands of a double helix using high temperatures or chemicals respectively.

As used herein, “variant helicase” means a variation from the nucleic acid or amino acid sequence of the helicase found in nature. A variation may be a mutation, a difference in glycosylation, a difference in primary, secondary, tertiary or quaternary protein structure, or a difference in other post-translational modifications commonly known in the art.

### EXAMPLES

### Example 1

### Creation of the RepΔ2B Variant Helicase

Buffers. Buffers were made with reagent-grade chemicals, using glass-distilled water that was further deionized using a Milli-Q System (Millipore). Buffer U is composed of 20 mM Tris.HCl, 6 mM NaCl, 1.7 mM MgCl₂, 5 mM 2-mercaptoethanol (2-ME), and 10% (vol/vol) glycerol (Aldrich), titrated to pH 7.5 at 25° C. SM buffer is 50 mM Tris HCl, 0.1 M NaCl, 8 mM MgSO₄, 0.01% gelatin, pH 7.5 at 25° C. C. Lysis buffer is 50 mM Tris HCl, 0.2 M NaCl, 20% (wt/vol) sucrose, 15% (vol/vol) glycerol, 1 mM EDTA, 2 mM 2-ME, pH 8.0 at 4° C. Buffer A is 50 mM Tris HCl, 0.5 M NaCl, 25% (vol/vol) glycerol, 2 mM 2-ME, pH 8.0 at 4° C. Buffer B is 50 mM Tris HCl, 20% (vol/vol) glycerol, 1 mM EDTA, 5 mM 2-ME, pH 7.5 at 4° C. Buffer C is 50 mM imidazole, 20% (vol/vol) glycerol, 1 mM EDTA, 5 mM 2-ME, pH 6.6 at 25° C. Storage buffer for all RepΔ2B protein is 100 mM Tris HCl, 50% (vol/vol) glycerol, 30 mM NaCl, 5 mM 2-ME, pH 7.5 at 4° C. Thrombin cleavage buffer is 40 mM Tris HCl, 19% (vol/vol) glycerol, 164 mM NaCl, 2.5 mM CaCl₂, 0.04 mg/ml PEG, 1 mM 2-ME, pH 8.4 at 22±2° C.

E. coli and Phage Strains and Plasmids. E. coli CK11Δrep, has the chromosomal copy of the rep gene deleted (Colasanti and Denhardt, (1987) Mol. Gen. Genet. 209:382-390). E. coli BL21 (DE3) and the plasmid pET28a were from Novagen. Φ X174 bacteriophage (strain K₉) was from P. Burgers (Washington University). The plasmid pRepO expresses wtRep protein under control of the temperature-inducible λP_{L} promoter (Lohman et al., J. Biol. Chem. 264:10139-47). The plasmid plWcl encodes the temperature-sensitive λcl857 repressor. The plasmid pGroESL (from C. Frieden, Washington University) overproduces the chaperonin GroES and GroEL.

RepΔ2B Plasmids. All restriction enzymes, T4 DNA ligase, and Pwo polymerase were purchased from Boehringer Mannheim or New England Biolabs, and were used under the conditions recommended by the suppliers. Oligodeoxynucleotides used in the PCR reactions were purchased from the Washington University Protein Nucleic Acid Chemistry Laboratory. Dye-terminated cycle sequencing was performed using an AmplitaqFS kit (PE Applied Biosystems) and analyzed on an ABI sequencer. Sequence data was analyzed using DNASTAR software (DNASTAR, Madison, Wis.). The sequences of the PCR primers used: GHG8, 5′- ATCTTTTCCGGCTCGAGTTATTTCCCTCGT (SEQ ID NO:3); GHG12, 5′- ACCGCCACCGCCACCGCCAGATATTTTGTA (SEQ ID NO:4); GHG13, 5′- CGGTGGC GGTGGCGGTGAGAGTGAAGAAGA (SEQ ID NO:5); GHG21, 5′- GAGCAATACACATATGCG TCTAAACCCCGGCCAA (SEQ ID NO:6); GHG25, 5′- GACCAGTCGATCTACTCCGCTCGC GGTGCACGTCCGCAA (SEQ ID NO:7).

Plasmids were constructed by established cloning methods (Ausubel et al., (1987) Current Protocols in Molecular Biology (Wiley, New York)). The N-terminal portion of the wtRep ORF from plasmid pRepO was amplified using primers GHG12 and GHG21, digested with NdeI and HindIII, and ligated into pET28a to generate subclone 1. The C-terminal portion of the wtRep ORF of pRepO was amplified using primers GHG8 and GHG25, and digested with HindIII and XhoI. This HindIII-XhoI restriction fragment was ligated into subclone 1 to create pGG206, encoding wtRep possessing an N-terminal hexa-histidine tag (+HRep). This histidine tag can be removed by cleavage with thrombin, yielding wtRep with an additional three amino acids (GSH) attached to its N terminus. A PCR overlap extension (Ho et al., (1989) Gene 77:51-9) was used to remove the codons encoding amino acids Thr-375 to Arg-542 in pGG206. Briefly, we generated two PCR fragments from the wtRep ORF: fragment 1 encodes the N-terminal portion of wtRep including domains 1A and 1B, and the N-terminal half of the 2A domain, and was amplified using primers GHG25 and GHG12; fragment 2 encodes the C-terminal half of Rep containing domain 2A, and was amplified using primers GHG13 and GHG8. Fragments 1 and 2 were then used in a third round of PCR to generate fragment 3, using primers GHG25 and GHG8. Fragment 3 was digested with HindIII and XhoI to yield a 714-bp fragment, which was ligated into pGG206, generating plasmid pGG205. Plasmid pGG205 encodes for +HRepΔ2B in which the 2B domain (Thr-375 to Arg-542) has been deleted and replaced with three Gly residues. The +HRepΔ2B has an N-terminal hexa-histidine tag with a thrombin cleavage site (indicated by the arrow): MGSSHHHHHHSSGLVPR↓GSH (SEQ ID NO:8). After thrombin cleavage, three extra residues (GSH) remain on the N terminus of −HRepΔ2B protein. To generate an untagged RepΔ2B ORF, pGG205 was digested with BstXI and MluI to yield a 683-bp fragment, which was cloned into pRepO digested with BstXI and MluI, generating plasmid pRepOΔ2B, which expresses RepΔ2B protein with an N terminus identical to wtRep, under the control of λP_{L} promoter. All ORFs were confirmed by DNA sequencing.

Protein Purification. Rep protein was purified to >99% homogeneity as described (Lohman et al., J. Biol. Chem. 264:10139-47) and its concentration determined spectrophotometrically (ε₂₈₀=7.68×10⁴ M⁻¹·cm⁻¹) (Amaratunga and Lohman, (1993) Biochemistry 32:6815-6820). His-tagged RepΔ2B (+HRepΔ2B) was purified from BL21 (DE3) cells carrying pGG205 and pGroESL. Histidine-tagged RepΔ2B (+HRepΔ2B) was purified from BL21 (DE3) cells carrying the plasmids pGG205 and pGroESL. Briefly, 1 liter of cells was used to inoculate 26 liters of terrific broth (Sambrook et al., (1989) Molecular Cloning: A Laboratory Manual (Cold Spring Harbor Lab. Press, Plainview, N.Y.) at 37° C. in a 40-liter BioFlow 5000 fermentor (New Brunswick Scientific, Edison, N.J.). Cells were grown to an OD₆₀₀ of 2.0, induced by addition of IPTG (300 m g/ml final concentration), and growth was continued at 37° C. for 2 h. The cells were then chilled and harvested by centrifugation. All procedures were done at 4° C. unless otherwise stated. Typically, 100 g of cell paste was resuspended in 500 ml of lysis buffer without EDTA. PMSF was added to a final concentration of 0.1 mM, and lysozyme was added to a final concentration of 0.4 mg/ml. After 1 h of gentle stirring at 4° C., the concentration of NaCl was adjusted to 0.5 M and the contents were subjected to pulsed sonication on ice for 15 min (50% duty cycle, power setting of 8). The cell lysate was clarified by a 14,000 rpm spin (JA14 rotor) for 1 h. The supernatant was then mixed with 40 ml of Ni-NTA agarose beads (Qiagen) pre-equilibrated in Buffer A. The mixture was gently stirred for 1 h, and the beads were collected by centrifugation (1,500 rpm for 10 min in a JA14 rotor). The beads were washed with 400 ml of buffer A and packed in a column (2.5 cm in diameter). The column was washed with Buffer A to baseline, followed by washing in steps with Buffer A plus 5 mM imidazole, 10 mM imidazole, and 25 mM imidazole (four column volumes each). +HRepΔ2B was eluted with Buffer A plus 150 mM imidazole, and the protein was precipitated from the eluate with ammonium sulfate at 350 g/liter (>>60% saturation). The ammonium sulfate pellet containing >>70% pure +HRepΔ2B was resuspended in Buffer B to a conductivity equivalent to 250 mM NaCl and loaded onto a single-stranded DNA (ssDNA) cellulose column (100 ml), pre-equilibrated in Buffer B plus 250 mM NaCl. The column was washed to baseline with Buffer B plus 250 mM NaCl and then washed with four column volumes of Buffer B plus 400 mM NaCl. +HRepΔ2B was eluted with Buffer B plus 3 M NaCl. The purity of +HRepΔ2B was greater than 98% at this stage and free of ssDNA exonuclease activity. To concentrate the protein, the eluate from the ssDNA column was dialyzed overnight vs. Buffer C plus 300 mM NaCl and then loaded onto a POROS 50 HS column (40 ml). The column was washed with Buffer C plus 300 mM NaCl, and the protein was eluted with Buffer C plus 1 M NaCl. The eluate was dialyzed vs. storage buffer, centrifuged at 40,000 rpm for 1 h, and flash frozen in 250-ml aliquots with liquid nitrogen. +HRepΔ2B protein concentration was determined spectrophotometrically in 6 M Guanidine HCl/20 mM Tris·Cl/0.1 M NaCl (pH 7.5) at 25° C., using an extinction coefficient, ε₂₈₀=4.58×10⁴ M⁻¹·cm⁻¹, calculated from its amino acid composition (4 Trp, 18 Tyr) (Gill and von Hippel (1989) Anal. Biochem. 182:319-26). The extinction coefficient of native +HRepΔ2B protein is ±280=(4.88±0.18)×10⁴ M⁻¹·cm⁻¹ in 10 mM Tris.Cl/500 mM NaCl/20% vol/vol glycerol (pH 8.3) at 25° C.

The histidine tag of +HRepΔ2B protein was cleaved to yield −HRepΔ2B by using restriction grade thrombin (Novagen) and −HrepΔ2B was purified as described. Typically, 1 mg of purified +HRepΔ2B was digested with 9.6 units of thrombin (unit definition from product manual) in 6 ml of cleavage buffer for at least 6 h on ice. After 6 h, >90% of +HRepΔ2B was cleaved, generating −HRepΔ2B. Longer incubation times did not generate any nonspecific cleavage as monitored by SDS/PAGE. The cleavage mixture was then passed through 1 ml of Ni-NTA agarose column pre-equilibrated in Buffer A plus 175 mM NaCl, the flow-through was collected and loaded directly onto a 1.5 ml ssDNA cellulose column pre-equilibrated in buffer B plus 250 mM NaCl. The column was washed with Buffer B plus 250 mM NaCl, and −HRepΔ2B protein (>98% pure) was eluted with Buffer B plus 3 M NaCl. The protein was concentrated by running it through a POROS 50 HS column, using the same protocol as for +HRepΔ2B, except that the protein was loaded onto the HS column in Buffer C plus 150 mM NaCl because we noticed that −HRepΔ2B was reproducibly eluted off the HS column at a lower salt concentration than +HRepΔ2B. The eluate was dialyzed vs. storage buffer, clarified by centrifugation at 40,000 rpm for 1 h, and flash frozen with liquid nitrogen.

RepΔ2B protein was purified from CK11Δrep/plWcl/pRepOΔ2B by using a slight modification of the wtRep purification (Lohman et al., J. Biol. Chem. 264:10139-47). The purification protocol of RepΔ2B is the same as that used for wtRep (Lohman et al., (1989) J. Biol. Chem. 264:10139-47) prior to ssDNA cellulose column step, except that RepΔ2B is not precipitated by Polymin P. Therefore, the supernatant from Polymin P precipitation was collected. RepΔ2B protein was precipitated from this supernatant by addition of ammonium sulfate (350 g/liter). The ammonium sulfate pellet was dissolved in Buffer C to a conductivity equivalent to 150 mM NaCl, and then loaded onto a POROS 50 HS column (40 ml) pre-equilibrated in Buffer C plus 150 mM NaCl. The column was washed with Buffer C plus 150 mM NaCl to baseline and then washed with Buffer C plus 250 mM NaCl (ten column volumes). The column was eluted with a 200-ml linear NaCl gradient (in Buffer C; 250 mM-1 M NaCl; five column volumes). RepΔ2B eluted at >) 500 mM NaCl. The fractions containing RepΔ2B were pooled and precipitated with 350 g/liter ammonium sulfate. The rest of the purification was identical to that described for the +HRepΔ2B purification, except for the ssDNA cellulose column wash step, in which Buffer B plus 400 mM NaCl was replaced by Buffer B plus 500 mM NaCl, and an additional ten column volumes of Buffer B plus 600 mM NaCl was used to wash the column to remove ssDNA exonuclease contaminant. RepΔ2B was eluted with Buffer B plus 3 M NaCl. At this stage, the RepΔ2B protein was >98% pure, with no detectable ssDNA exonuclease activity.

### Example 2

### Unwinding Kinetics

DNA Substrates. Oligodeoxynucleotides were synthesized and purified to >99% homogeneity and their concentrations determined as described (Cheng et al., (2001) J. Mol. Biol. 310:327-350). Oligodeoxynucleotides were radiolabeled with ³²P at the 5′ end by using T4 polynucleotide kinase (United States Biochemical), and purified as described (Ali et al., (1999) J. Mol. Biol. 293:815-34). DNA substrates contain an 18-bp duplex region with a 3′-(dT)₂₀ ssDNA tail (all oligothymidylates) and were prepared as described (Cheng et al., (2001) J. Mol. Biol. 310:327-350). The base sequence of the “top strand” of the 18-bp duplex is 5′-GCCTCGCTGCCGTCGCCA-3′ (SEQ ID NO:9) and is the same as used in previous experiments (Cheng et al., (2001) J. Mol. Biol. 310:327-350).

Single-Turnover (STO) DNA Unwinding Kinetics. STO DNA unwinding experiments were carried out using a quenched-flow apparatus (RQF-3, Kintek, University Park, Pa.) at 25.0° C. in Buffer U, essentially as described (Cheng et al., (2001) J. Mol. Biol. 310:327-350). Briefly, helicase protein was preincubated with ³²P-labeled DNA substrate (2 nM) in Buffer U containing BSA (0.1 mg/ml) on ice for 20 min, and then loaded in one loop of the quenched-flow apparatus. The other loop contained ATP (3 mM) and an excess of an unlabeled 10-bp hairpin DNA (8 μM) possessing a 3′-(dT)₄₀ tail (mock DNA substrate, HP10T40) in Buffer U that served as a trap to prevent rebinding of free protein to the DNA substrate. Control experiments indicated that a final concentration of 4 μM HP10T40 is sufficient to trap 800 nM free protein. This insures that these are STO DNA unwinding experiments (single round of DNA unwinding). The DNA and protein concentrations reported are the preincubation concentrations before mixing with ATP. Reactions were initiated by rapidly mixing the two solutions and then quenched by addition of 400 mM EDTA in 10% (vol/vol) glycerol at time intervals from 2 ms to 120 s after mixing. Quenched samples were analyzed after separation of the duplex substrate from the ssDNA product by electrophoresis in nondenaturing 10% polyacrylamide gels (PAGE). The radioactivity in each band was quantitated using a Storm 840 Phosphorlmager (Molecular Dynamics). DNA unwinding time courses were analyzed using Eq. 1, derived from Scheme 1 (Cheng et al., (2001) J. Mol. Biol. 310:327-50; A. L. Lucius, N. K. Maluf, C. J. Fischer, and T. M. L., unpublished work), where F(t) is the fraction of DNA molecules unwound at time t, A_{T} is the total amplitude of DNA unwinding, n is the number of steps in the unwinding reaction, and k_{obs}=(kᵤ+k_{d}).

$$F ( t ) = {A}_{T} ( ( 1 - {\sum }_{r = 1}^{n} \frac{{( {k}_{obs} t )}^{r - 1}}{( r - 1 ) !} \cdot {ⅇ}^{( - {k}_{obs} t )} ) - {ⅇ}^{- {k}_{np} t} ( 1 - x ) {( \frac{{k}_{obs}}{{k}_{obs} - {k}_{np}} )}^{n} \qquad Eq . 1$$

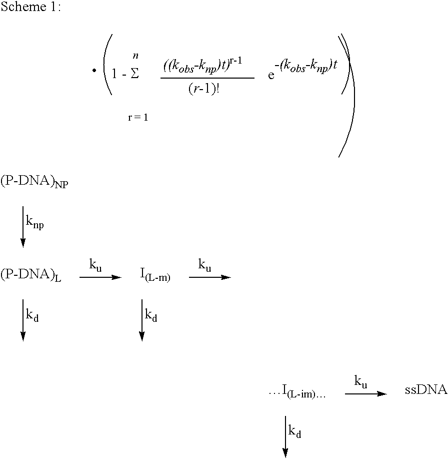

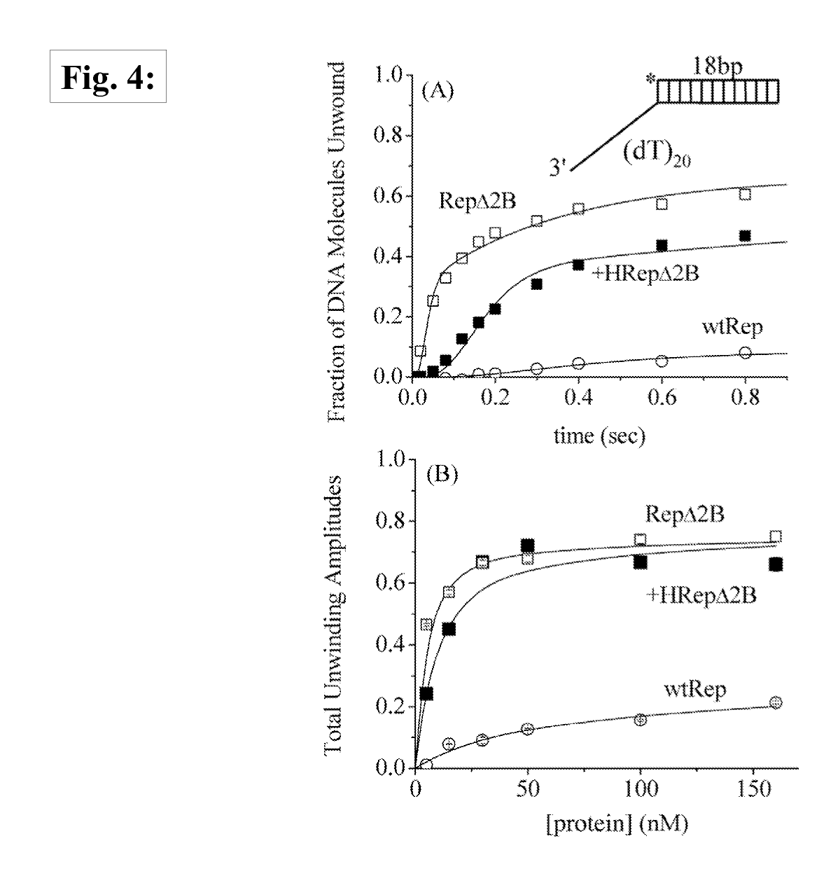

DNA Helicase Activity of RepΔ2B in Vitro. Three versions of the RepΔ2B protein were purified and characterized: +HRepΔ2B with a histidine tag added to the N terminus, −HRepΔ2B obtained after thrombin cleavage of the histidine tag from +HRepΔ2B (which retains three additional amino acids, GSH, fused to the N terminus), and RepΔ2B without any tag. All three were purified to >98% purity, and their helicase activities were compared with wtRep protein by performing STO DNA unwinding kinetics experiments (Ali et al., (1997) Science 275:377-80), using an 18-bp DNA substrate with a 3′-(dT)₂₀ tail, as depicted in FIG. 4. In these STO experiments, only protein that is prebound to the DNA can participate in DNA unwinding. Although the quenched-flow assay is an “all or none” DNA unwinding assay, detecting only fully unwound DNA, the time courses of these experiments can yield the rates of the rate-limiting steps that are repeated during the course of DNA unwinding (Ali et al., (1997) Science 275:377-80).

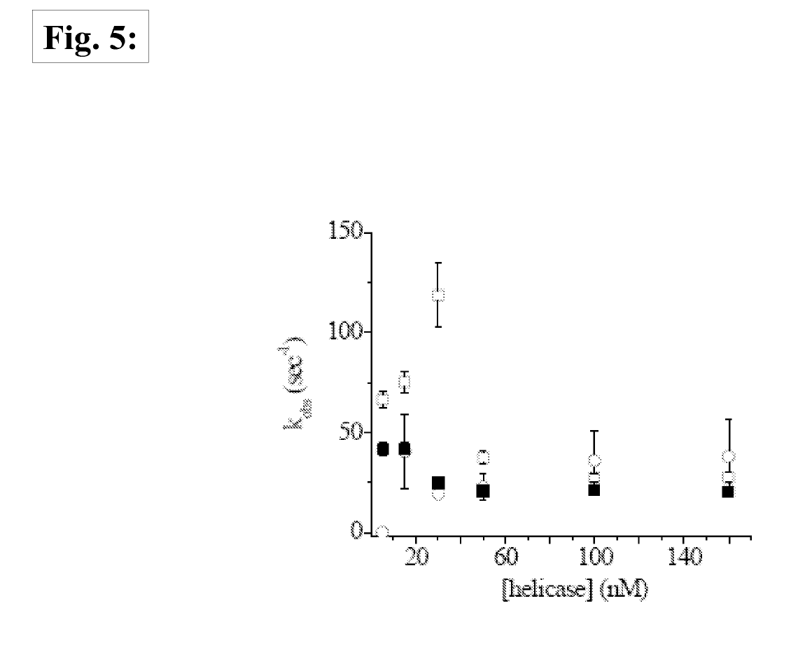

FIG. 4A shows individual time courses for wtRep, +HRepΔ2B, and RepΔ2B at a protein concentration of 30 nM. The results with −HRepΔ2B were identical to those obtained with RepΔ2B (data not shown). All time courses were biphasic, as observed previously for both UvrD (Ali et al., (1999) J. Mol. Biol. 293:815-34; Ali et al., (1997) Science 275:377-80) and wtRep (Cheng et al., (2001) J. Mol. Biol. 310:327-50). The lag phase reflects the transient formation of partially unwound DNA intermediates along the path to complete DNA unwinding, and depends on the duplex DNA length. The presence of the second, slower phase suggests that the helicase protein is prebound to the DNA substrate in at least two different states, consistent with our previous experiments (Ali et al., (1999) J. Mol. Biol. 293:815-34). These time courses were analyzed using Scheme 1 (shown above), which assumes that the helicase can be bound to the DNA in two forms, a productive from, (P-DNA)L, and a nonproductive from, (P-DNA)_{NP}. The productively bound form unwinds DNA in a series of rate-limiting steps with rate constant kᵤ. The nonproductively bound helicase must first isomerize with rate constant k_{NP} to form the productive form, which can then initiate DNA unwinding. Because we have not yet performed experiments as a function of duplex length with these helicases, which is necessary to estimate the number of intermediate steps, n, needed to fully unwind the 18-bp duplex (see Scheme 1), we have analyzed each time course by assuming a value of n=4, based on our studies of DNA unwinding by E. coli UvrD (Ali et al., (1999) J. Mol. Biol. 293:815-34; Ali et al., (1997) Science 275:377-80), which is structurally homologous to wtRep. The solid lines describing these time courses were generated using the best fit parameters obtained from nonlinear least squares fitting of each unwinding time course to Eq. 1. FIG. 4 shows that both the observed rates and the extents of unwinding for RepΔ2B and +HRepΔ2B are significantly greater than for wtRep. The values for k_{obs} (=kᵤ+k_{d}; assuming n=4) are 118.8±16 s⁻¹ for RepΔ2B, 42.0±3.2 s⁻¹ for +HRepΔ2B, and 17.9±5.4 s⁻¹ for wtRep. Interestingly, unwinding by RepΔ2B is also faster than +HRepΔ2B, which indicates that the histidine tag has a slightly inhibitory effect under these conditions. At protein concentrations above 100 nM, the observed rates of unwinding by RepΔ2B decrease and become similar to those observed for wtRep at 17±5.4 s⁻¹ (FIG. 5). This effect of protein concentration is currently under study, but may indicate an effect of different oligomeric forms of RepΔ2B on DNA unwinding. FIG. 4B shows the dependence of the extent of DNA unwinding (fraction of DNA molecules unwound) on helicase concentration. The total amplitudes of DNA unwinding (fast and slow phases) for both +HRepΔ2B and RepΔ2B are much greater than for wtRep at all protein concentrations. The midpoint concentrations obtained from fitting the unwinding amplitudes to a hyperbola are also much lower for RepΔ2B (3.5±0.6 nM) and +HRepΔ2B (8.5±3.0 nM), compared with wtRep (57±20 nM).

### Example 3

### In Vivo Experiments

Plaque Assay for Φ X174 Phage. Φ X174 plaque assays were performed with Escherichia coli CK11Δrep/plWcl, as well as CK11Δrep/plWcl/pRepO, and CK11Δ rep/plWcl/pRepOΔ 2B. Phage Φ X174 (strain K9) was stored in 50 mM Na₂B₄O₇ at 4° C., and serial dilutions were made in SM buffer immediately prior to experiments. Briefly, 300 μl of a fresh bacterial culture grown to saturation at 30° C. was gently mixed with 100 μl of a diluted phage stock in a 5-ml sterile culture tube, and incubated at 25° C. for 20 min. The tubes were then shifted to a 37° C. heat block for an additional 10 min and then plated on X plates at 37° C. after adding 2.5 ml of top-agar, pre-equilibrated at 45° C. The plates were incubated at 37° C. Typically plaques started to appear after 4 h and were counted after 9-10 h at 37° C.

Bacterial Growth Curves. Bacterial growth curves were determined for the following E. coli cells: CK11Δrep/plWcl, CK11Δrep/plWcl/pRepO, and CK11Δrep/plWcl/pRepOΔ2B. Briefly, 1 ml of cells in early exponential growth at 30° C. was diluted into 50 ml of LB broth in a 250-ml flask so that each flask had the same initial optical density at 600 nm (OD₆₀₀). The flasks were then shaken at 300 rpm in an incubator at 30° C. OD₆₀₀ readings were taken as a function of time by diluting cells into LB broth prechilled to 4° C.

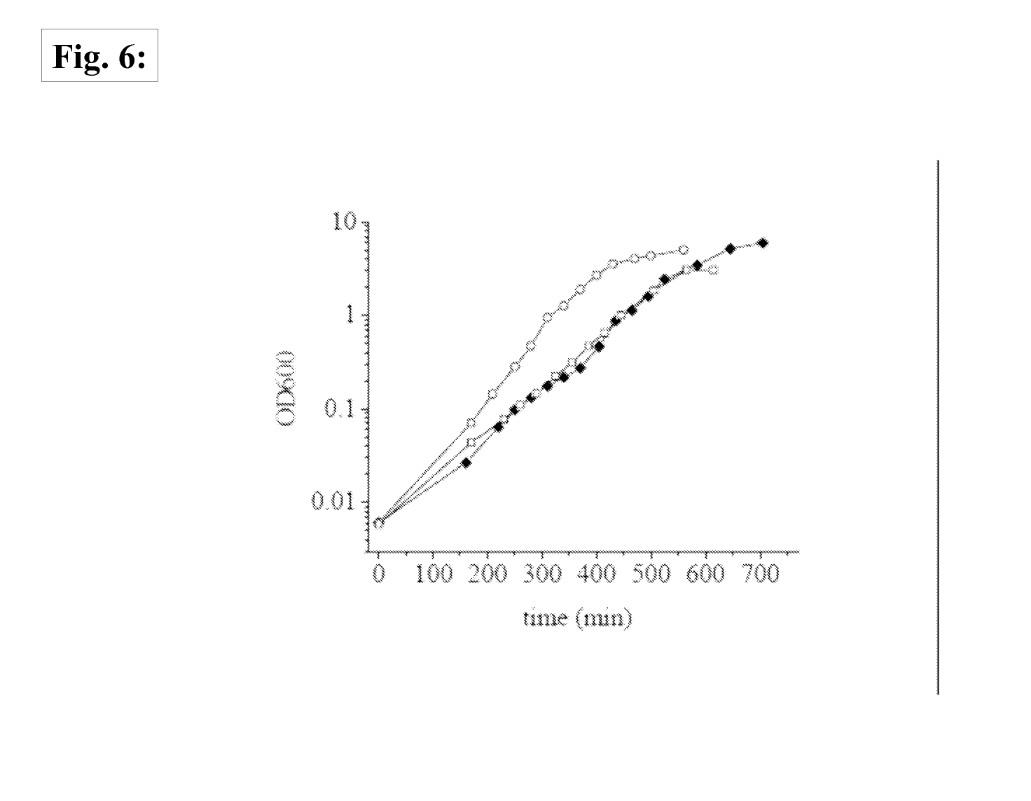

Effect of RepΔ2B on E. coli Growth. Growth rates were determined for CK11Δrep/plWcl/pRepOΔ2B, CK11Δrep/plWcl/pRepO, and CK11Δrep/plWcl at 30° C. in LB media, conditions under which overexpression of Rep and RepΔ2B has not been induced. CK11Δrep/plWcl/pRepOΔ2B has a generation time of 58±4 min, which is slower than that of CK11Δrep/plWcl/pRepO (40±4 min), but the same as that of CK11Δrep/plWcl alone (FIG. 6).

RepΔ2B Protein Supports Φ X174 Phage Replication. Although Rep protein is not essential for the viability of E. coli under laboratory growth conditions, Rep protein is the only E. coli helicase that functions to unwind the double-stranded replicative form of Φ X174 phage DNA required for replication of that phage (Denhardt et al., (1967) PNAS 57:813-20; Tessman and Peterson (1976) J. Virol. 20:400-12; Scott et al., (1977) PNAS 74:193-7). We therefore tested whether RepΔ2B protein can support Φ X174 DNA replication in vivo by performing X174 phage plaque assays, using CK11Δrep/plWcl, which carries a deletion of the wild-type rep gene, as the host. As summarized in Table 2, no plaques were formed when CK11Δrep/plWcl was used, reflecting the inability of ΦX174 phage to replicate in a rep⁻E. coli host.

TABLE 2
ΦX174 plaque assay

| Phage dilution | No. of plaques (pRepO) | No. of plaques (pRepOΔ2B) |
|---|---|---|
| 10⁻⁶ | 71 ± 10 | 60 ± 8 |
| 10⁻⁷ | 7 ± 2 | 6 ± 2 |

The plaque numbers are averaged from eight independent assays.

However, CK11Δrep/plWcl carrying either of the plasmids, pRepO or pRepOΔ2B, encoding wtRep protein or the RepΔ2B protein, respectively, produced essentially the same numbers of plaque-forming units (pfu). The only difference was that the plaque size was slightly smaller when assayed with CK11Δrep/plWcl containing pRepΔ2B than with the same host containing pRepO. This likely reflects the fact that the growth rate of CK11 Δrep/plWcl/pRepΔ2B is slower than the growth rate of CK11Δrep/plWcl/pRepO (see below). Nonetheless, these results indicate that RepΔ2B can function as a helicase in Φ X174 DNA replication in vivo.

### Example 4

### Sedimentation Equilibrium Experiments

Sedimentation equilibrium experiments were performed using an Optima XL-A analytical ultracentrifuge (Beckman Instruments, Fullerton, Calif.) and analyzed as described (Maluf et al., (2003) J. Mol. Biol. 325:889-912). Apparent molecular weights were obtained by nonlinear least-squares (NLLS) fitting of the concentration profiles to Eq. 2,

$${A}_{t} = {\sum }_{i = 1}^{n} exp ( ln {A}_{0 , i} + {σ}_{i} ( {r}^{2} - {r}_{ref}^{2} ) / 2 ) + b$$

where A_{T} is the total absorbance at radial position r; A_{0,i} is the absorbance of component i at reference radial position (r_{ref}); b is the baseline offset;
σᵢ=[Mᵢ(1−νᵢρ)ω²]/RT; Mᵢ is molecular mass of component i; νᵢ is the partial specific volume of component i; ρ is the solution density; w is the angular velocity; R is the gas constant; and T is absolute temperature. ρ was calculated based on the buffer composition by using the program SEDNTERP (Laue, T. M. (1992) Ultracentrifugation in Biochemistry and Polymer Science (Royal Society of Chemistry, London). Global NLLS fit of the absorbance profiles to Eq. 2 returns M for each component present in equilibrium, providing υᵢ is known or calculated. The NLLS fitting program WINNONLIN (Johnson et al., (1981) Biophys. J. 36:575-588) was used for analysis of a single-species system (i.e., n=1 in Eq. 2), whereas CONLIN (Williams, D. J. & Hall, K. B. (2000) Methods Enzymol. 321:330-352) was used for NLLS analysis of multiple species (n=2 in Eq. 2). Sedimentation equilibrium data obtained for wild-type Rep (wtRep) and RepΔ2B were well described by a single ideal species (n=1 in Eq. 2). υ for wtRep and RepΔ2B were calculated from the known amino acid composition by using SEDNTERP. The computed values of υ were then corrected for the presence of 20% (vol/vol) glycerol in the buffer as described (Maluf, N. K. & Lohman, T. M. (2003) J. Mol. Biol. 325:889-912). The slight differences between the experimentally determined and predicted molecular masses for wtRep and RepΔ2B likely reflect deviations of the actual partial specific volumes from those calculated from the amino acid composition assuming additivity (0.746 ml/g for RepΔ2B and 0.745 ml/g for wtRep). If we assume that the molecular masses calculated from amino acid composition are correct, then we calculate values of 0.767 (±0.005) ml/g and 0.762 (±0.008) ml/g at 25° C. for the partial specific volumes of RepΔ2B and wtRep, respectively. Sedimentation equilibrium experiments for RepΔ2B and wtRep were performed without 2-mercaptoethanol to eliminate the absorbance effect of the reducing agent at 230 nm. Experiments repeated in the presence of 1 mM 2-mercaptoethanol gave identical results.

Sedimentation equilibrium profiles of wtRep and RepΔ2B in the presence of a molar excess of DNA I were well described by two exponential terms (n=2 in Eq. 2), representing free DNA and the protein-DNA complex. DNA I alone in Buffer M (200 mM NaCl) sediments as a single ideal species with M=17.6 (+0.4) kDa (υ_{DNA} for free DNA was assumed to be equal to 0.58 ml/g) (Ralston, G. (1993) Introduction to Analytical Ultracentrifugation (Beckman Instruments, Fullerton, Calif.)), which is in agreement with M calculated from nucleotide composition and M of Cy3 fluorophore (M=17.6 kDa). For the NLLS analysis of the protein-DNA samples, σ_{DNA} was fixed to the value determined independently for DNA I, while floating for σ_{protein-DNA}. The values of υ for the protein-DNA complexes were calculated as the weight-averaged sum of υ for the individual components.

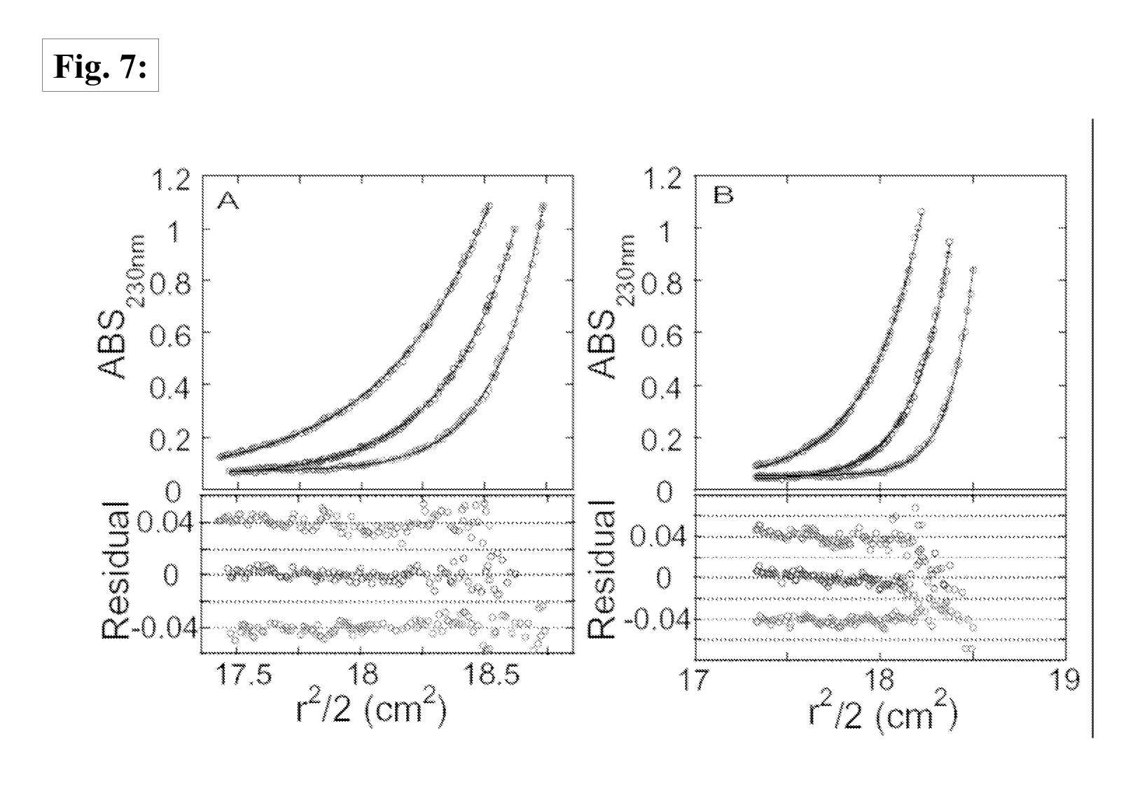

The assembly states of RepΔ2B alone and when bound to a short DNA substrate were examined by sedimentation equilibrium in Buffer M [20 mM Mops/20% (vol/vol) glycerol, pH 6.5] plus 200 mM NaCl at 25° C. Experiments performed at three RepΔ2B concentrations (0.5-2 μM) at rotor speeds of 23,000, 28,000, and 34,000 rpm in the absence of DNA (FIG. 7A) showed that RepΔ2B behaved as a single ideal species with molecular mass, M=57.4 (±2.3) kDa, indicating that RepΔ2B is monomeric under these conditions. Sedimentation equilibrium experiments performed under the same solution conditions indicate that wtRep is monomeric with M=76.8 (4.8) kDa (FIG. 7B). Therefore, both wtRep and RepΔ2B exist as monomers in Buffer M plus 200 mM NaCl at 25° C., at least up to concentrations of 2 μM, in agreement with previous studies of wtRep (Chao and Lohman (1991) J. Mol. Biol. 221:1165-81).

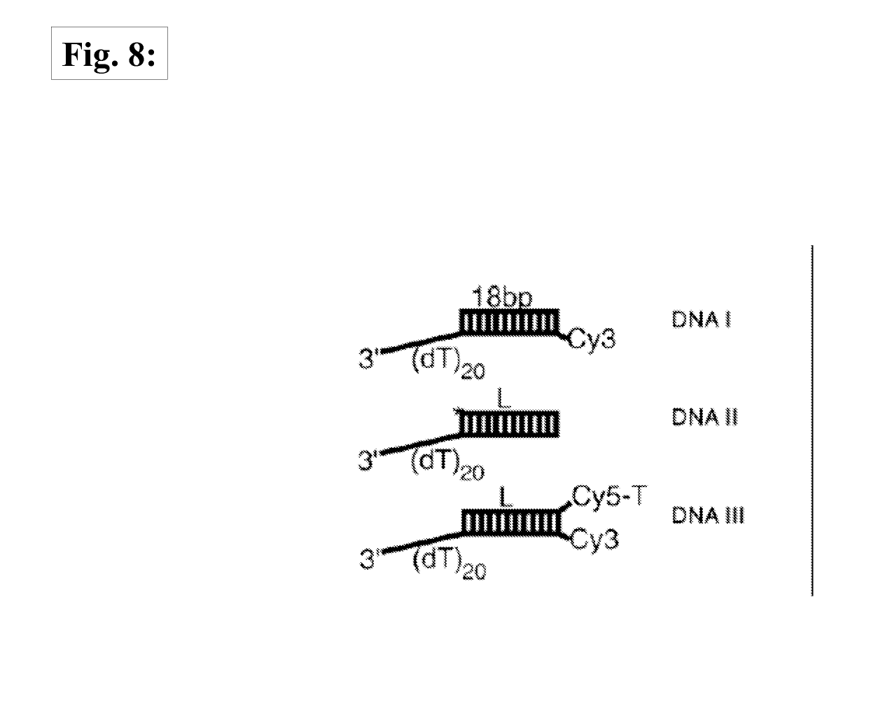

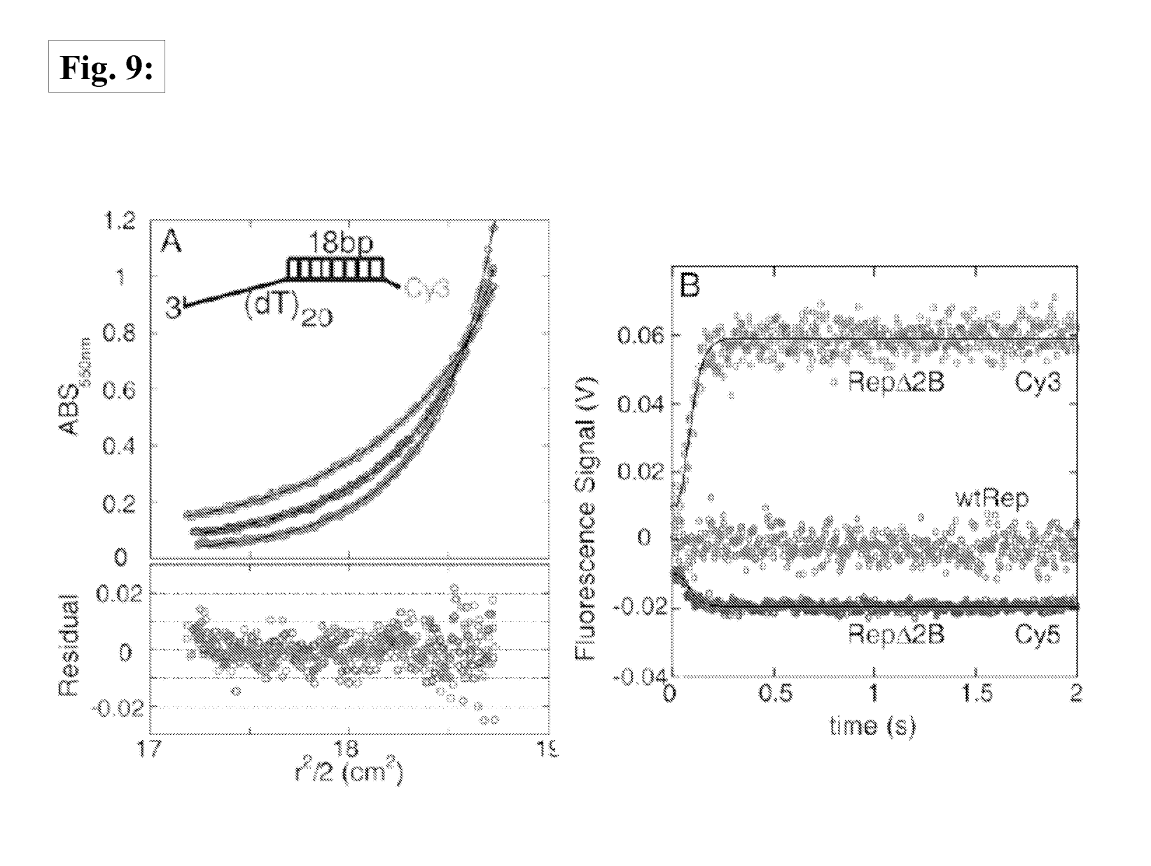

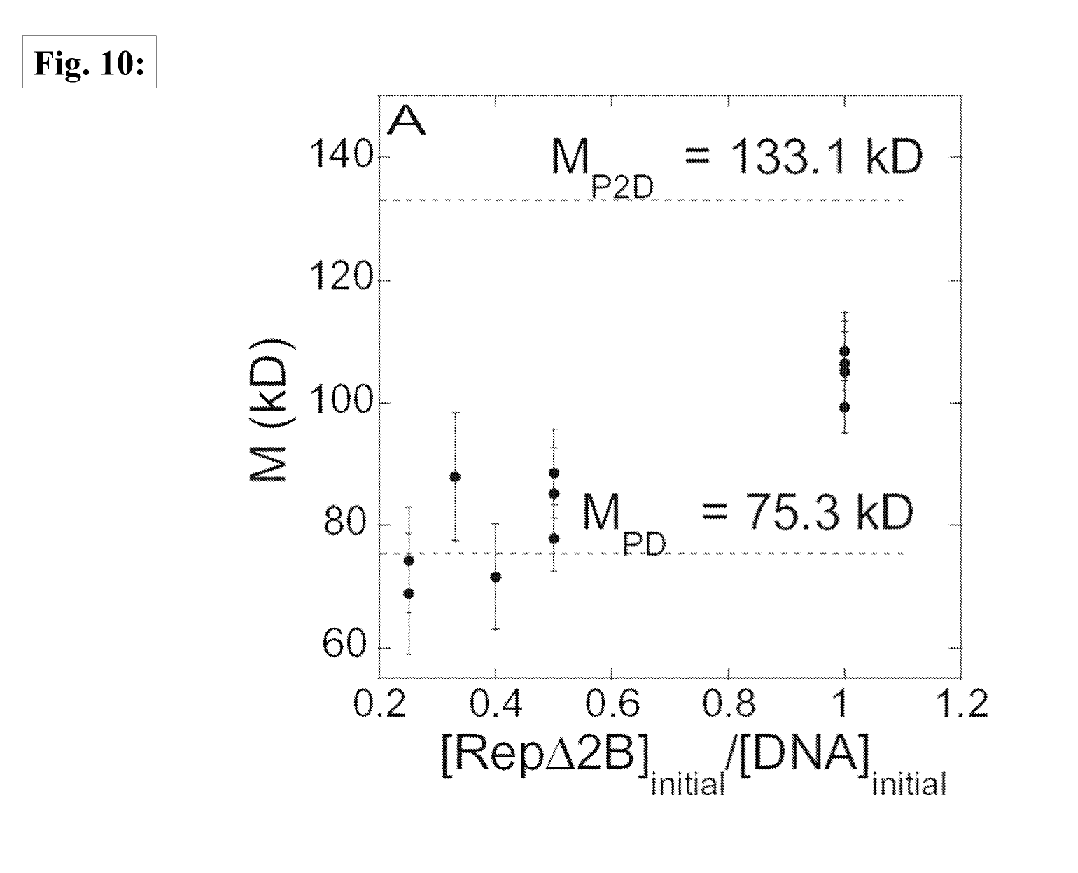

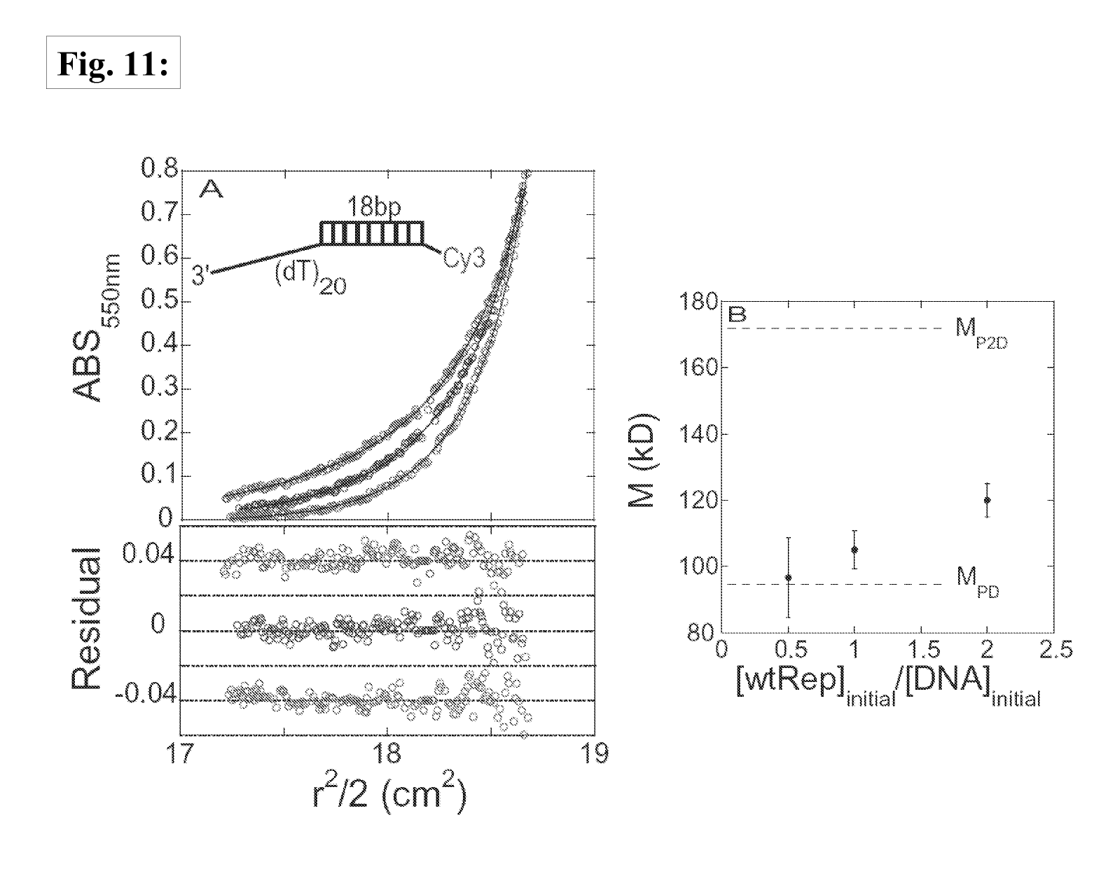

To examine the helicase activity of RepΔ2B monomers, STO DNA unwinding experiments were performed under conditions such that no more than one RepΔ2B monomer is prebound to each DNA substrate. To determine these conditions, we performed sedimentation equilibrium experiments by using an 18-bp DNA substrate containing a 3′-(dT20) ssDNA tail and a Cy3 chromophore attached covalently to its blunt end via the 5′ end of one strand of the DNA duplex (DNA I; FIGS. 8 and 9A; Table 1). By monitoring Cy3 absorbance (550 nm), the DNA concentration profile can be observed without interference from the RepΔ2B. Sedimentation equilibrium experiments were performed at different ratios of RepΔ2B to DNA (Buffer M plus 200 mM NaCl at 25° C.), and FIG. 9A shows a representative set of results (2 μM RepΔ2B and 4 μM DNA). NLLS analysis of the absorbance profiles indicates two DNA species with molecular masses, M=17.6 (±0.4) kDa and 77.9 (±5.4) kDa, corresponding to free DNA and DNA bound by one RepΔ2B monomer, respectively. Experiments at varying RepΔ2B/DNA molar ratios indicate that as long as the input molar ratio of RepΔ2B/DNA <0.6, no more than one RepΔ2B is bound per DNA substrate at equilibrium. (FIG. 10) Experiments performed with wtRep protein in Buffer M plus 200 mM NaCl at 25° C. (FIG. 11) showed that no more than one wtRep is bound per DNA substrate at wtRepΔDNA <1.

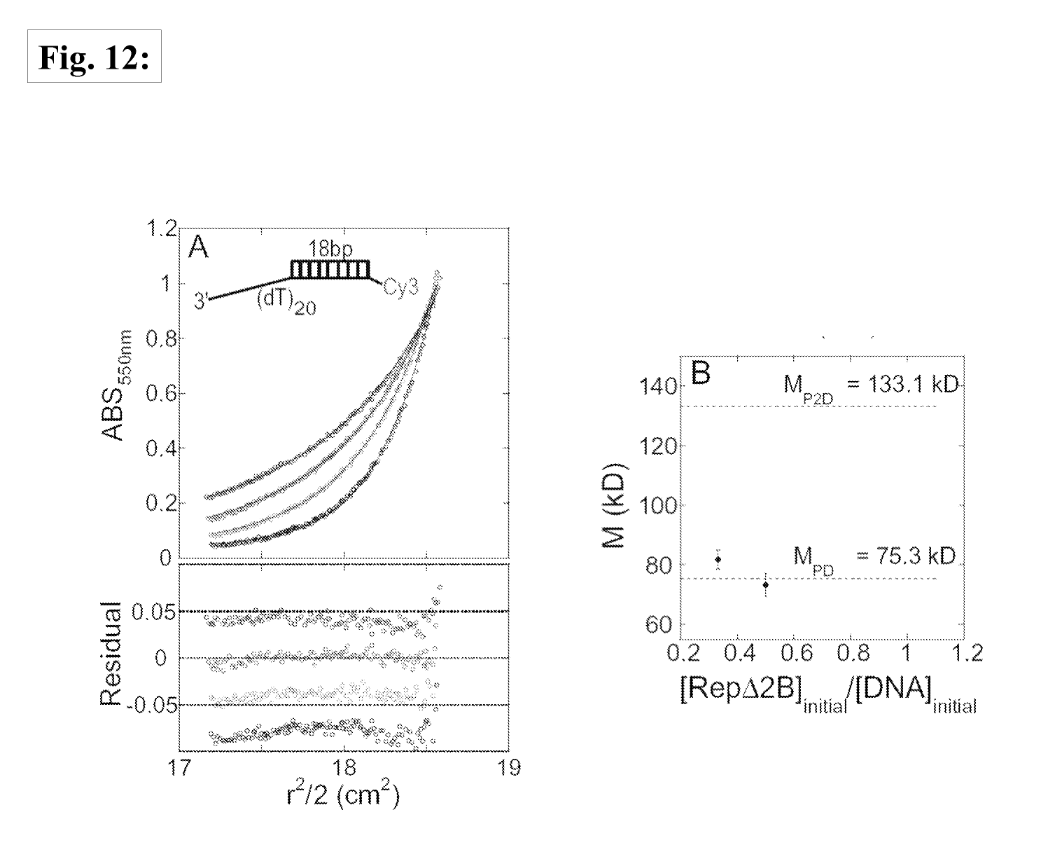

We were unable to characterize the assembly state(s) of the RepΔ2B protein alone at NaCl concentrations below 200 mM by using sedimentation equilibrium, because stable equilibrium distributions of protein were not obtainable at these lower [NaCl]. However, RepΔ2B-DNA complexes could be examined at 50 mM NaCl in the presence of a molar excess of DNA, and we find that RepΔ2B binds to the DNA substrate as a monomer when the DNA is in molar excess over RepΔ2B. (FIG. 12)

### Example 5

### Single-Stranded DNA (ssDNA) Translocation Experiments

Rates of ssDNA translocation by wtRep and RepΔ2B monomers were determined as described (Fischer et al., (2004) J. Mol. Biol. 344:1287-1309). Briefly, a fluorophore (Cy3) is attached to either the 3′ or the 5′ end of a series of synthetic oligodeoxythymidylates (dTL) of varying lengths, L, and arrival of the protein at the end of the DNA is monitored by the increase in Cy3 fluorescence as the protein interacts with the Cy3. By using a molar excess of DNA (200 nM), monomers of wtRep or RepΔ2B (50 nM) were prebound to the DNA labeled at either the 3′ or the 5′ end with Cy3 and then rapidly mixed with a solution containing 3 mM MgCl₂, 3 mM ATP, and 8 mg/ml heparin (Buffer M plus 200 mM NaCl, 25° C.). The heparin serves as a trap for free protein, thus preventing rebinding of free protein to the DNA upon dissociation and ensuring translocation reactions that are STO (single round) (Fischer et al., (2004) J. Mol. Biol. 344:1287-1309).

Nonlinear least squares (NLLS) analyses of the translocation time courses were performed as described (Fischer et al., (2004) J. Mol. Biol. 344:1287-1309) using Eq. 3 (RepΔ2B; Scheme 2) or Eq. 4 (wtRep; Scheme 3):

$$\begin{matrix}f ( t ) = D + \frac{A}{1 + nr} {L}^{- 1} ( \frac{1}{s + {k}_{c}} ( 1 + \frac{{k}_{t} r}{s + {k}_{d}} ( 1 - {( \frac{{k}_{t}}{s + {k}_{t} + {k}_{d}} )}^{n} ) ) ( 1 + \frac{{Bk}_{c}}{s + {k}_{end}} ) ) & Eq . 3 \\ f ( t ) = D + \frac{A}{1 + nr} {L}^{- 1} ( \frac{1}{s + {k}_{a}} ( 1 + \frac{{k}_{t} r}{s + {k}_{d}} ( 1 - {( \frac{{k}_{t}}{s + {k}_{t} + {k}_{d}} )}^{n} ) ) ( 1 + \frac{{Ck}_{c}}{s + {k}_{end}} + \frac{{Bk}_{a}}{s + {k}_{c}} ) ) & Eq . 4\end{matrix}$$

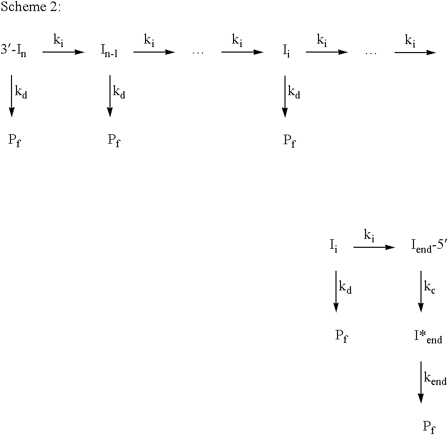

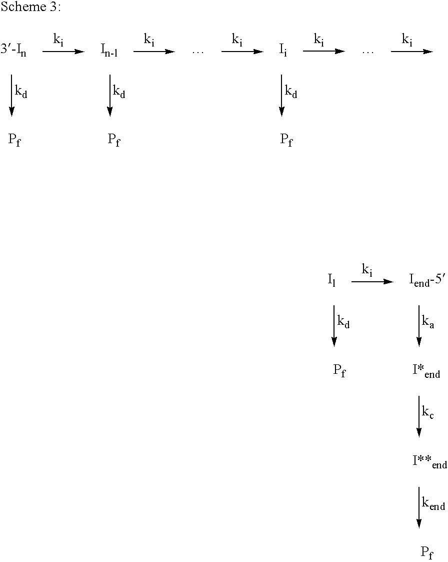

where L⁻¹ is the inverse Laplace transform operator, kₜ is the rate constant for the ratelimiting step during translocation, k_{d} is the rate constant for dissociation from internal regions of the ssDNA; k_{c} and k_{end} are the rate constants for the two-step dissociation from the 5′ end of the DNA; n is the maximum number of steps required for a protein monomer bound at the 3′ end of the ssDNA to reach the 5′ end of the DNA; A=f_{end}I(0); I(0) is the total concentration of protein bound to the DNA at time 0; f_{end} is the fluorescence signal associated with a protein monomer bound at the 5′ end of the DNA in state I_{end}; B=f*_{end}/f_{end}; f*_{end} is the fluorescence signal associated with a protein monomer bound at the 5′ end of the DNA in state I*_{end}; C=**_{end}/f_{end}; f**_{end} is the fluorescence signal associated with a protein monomer bound at the 5′ end of the DNA in state I**_{end} (only in Eq. 4); D is a scalar offset; and r=I_{1≦i≦n}(0)/I_{end}(0) is the ratio at time 0 of the concentration of protein bound to any position other than the 5′ end of the DNA to the concentration of protein bound at the 5′ end of the DNA (Fischer & Lohman, (2004) J. Mol. Biol. 344:1265-86).

Values of the dissociation rate constants, k_{d}, used in the NLLS analysis of translocation time courses according to Schemes 2 and 3 (Eqs. 3 and 4, respectively) were constrained to the values of k_{d,obs} determined in experiments monitoring the dissociation of wtRep or RepΔ2B during translocation along poly(dT) upon addition of heparin (4 mg/ml, final concentration) as a trap for free protein (Fischer & Lohman, (2004) J. Mol. Biol. 344:1287-1309). These values of k_{d,obs} (Buffer M, 25° C.) are for wtRep: 0.465 (±0.002) s−1 at 50 mM NaCl and 0.436 (±0.007) s−1 at 200 mM NaCl, and for RepΔ2B: 0.91 (±0.04) s−1 at 50 mM NaCl and 0.798 (±0.005) s−1 at 200 mM NaCl. The poly(dT) sample had a weight-averaged sedimentation coefficient, s_{20,w}=9.14 (±0.1) S, as determined from boundary sedimentation velocity experiments (Fischer & Lohman, (2004) J. Mol. Biol. 344:1287-1309). This corresponds to an average length of 886 (±21) nucleotides (Fischer & Lohman, (2004) J. Mol. Biol. 344:1287-1309). As discussed (Fischer & Lohman, (2004) J. Mol. Biol. 344:1265-86), due to the finite length of the poly(dT), these values of k_{d,obs} represent slight overestimates of the true values of k_{d}, although the macroscopic translocation rates reported here are not significantly affected by the uncertainties in k_{d}. The value of k_{d,obs} for wtRep was linearly dependent on heparin concentration in the concentration range used in our experiments, indicating that heparin actively displaces wtRep during its translocation, as was observed with UvrD (Fischer & Lohman, (2004) J. Mol. Biol. 344:1265-86); however, the value of k_{d,obs} for RepΔ2B was found to be independent of heparin concentration. This suggests that an interaction between heparin and the 2B subdomain of wtRep is involved in the mechanism of heparin-dependent displacement of wtRep from ss-DNA, implying that the 2B subdomain possesses affinity for heparin. We note that although inclusion of the additional step with rate constant kₐ in Scheme 3 does result in a significant reduction in the variance of the NLLS fit of wtRep translocation data compared to use of Scheme 2, the value of the macroscopic translocation rate reported here (mkₜ) is not sensitive to the inclusion of this additional step in the scheme for translocation.

ssDNA Translocation of wtRep and RepΔ2B Monomers.

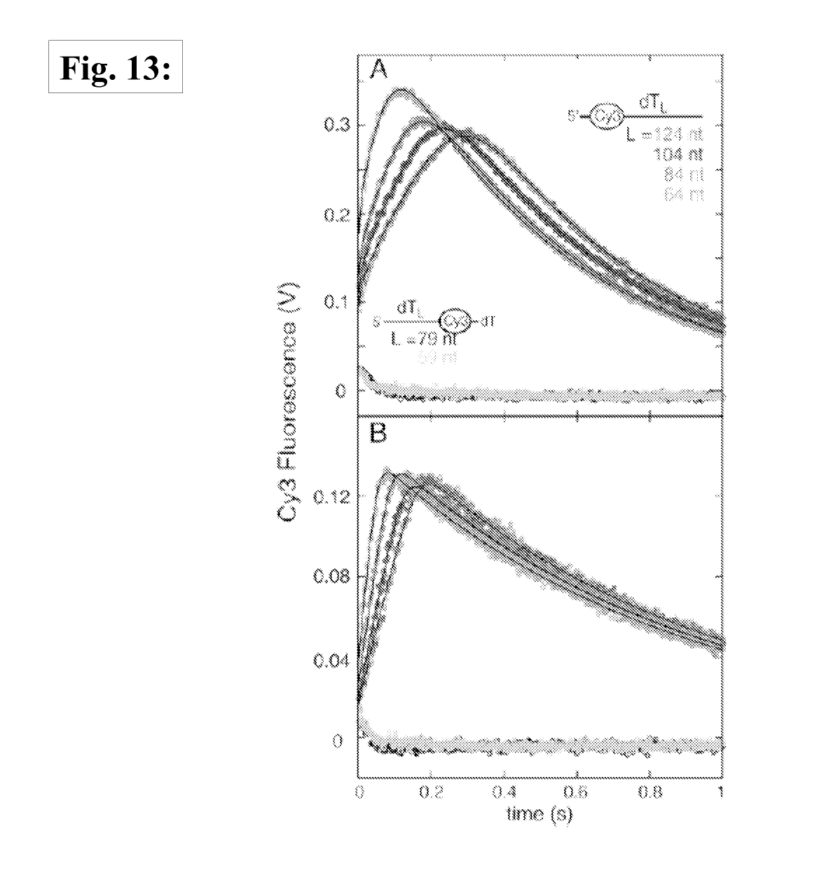

Next examined was the ability of wtRep and RepΔ2B monomers to translocate along ssDNA to determine whether the lack of helicase activity of a wtRep monomer reflects an inability of the monomer to translocate. A fluorescence stopped flow assay was used as described (Fischer et al., (2004) J. Mol. Biol. 344:1287-1309; Fischer and Lohman (2004) J. Mol. Biol. 344:1265-86). The resulting time courses for wtRep monomers (FIG. 13A) and RepΔ2B monomers (FIG. 13B) on dT_{L} (L=64, 84, 104, and 124 nucleotides), labeled with Cy3 at the 5′ end, show an initial ATP-dependent enhancement of Cy3 fluorescence associated with the accumulation of protein at the 5′ end of the DNA, followed by a slower decrease in fluorescence reflecting protein dissociation from the 5′ end of the DNA. Furthermore, the peak in the Cy3 fluorescence time course moves to longer times as the length of the DNA increases. In contrast, control experiments performed with dT_{L} (L=59 and 79 nucleotides), labeled with Cy3 at the 3′ end, show a length-independent decrease in Cy3 fluorescence (FIG. 13). These results indicate that both wtRep and RepΔ2B monomers translocate with biased 3′ to 5′ directionality along ssDNA in ATP-dependent reactions (Fischer and Lohman (2004) J. Mol. Biol. 344:1265-86). As described (Fischer et al., (2004) J. Mol. Biol. 344:1287-1309), one can analyze the time courses in FIG. 13 to obtain quantitative estimates of the macroscopic rate of translocation by using the minimal sequential “n-step” mechanism shown in Scheme 2, by using Eq. 3. The macroscopic translocation rate determined from NLLS analysis of RepΔ2B monomer translocation time courses (FIG. 13B) is 530 (±10) nucleotides s⁻¹ (Buffer M, 200 mM NaCl, 25° C.). Interestingly, the minimal sequential “n-step” mechanism needed to describe the wtRep translocation time courses requires the inclusion of an additional step (kₐ in Scheme 3). The macroscopic translocation rate determined from NLLS analysis of wtRep monomer translocation time courses (FIG. 13A) according to Scheme 3 (Eq. 4) is 298 (2) nucleotides s⁻¹ (Buffer M, 200 mM NaCl, 25° C.). Translocation experiments performed at 50 mM NaCl (Buffer M, 25° C.) gave similar results (630 (±20) nucleotides s⁻¹ for RepΔ2B monomers vs. 279 (±2) nucleotides s⁻¹ for wtRep monomers). Therefore, both wtRep and RepΔ2B monomers can translocate rapidly and with 3′ to 5′ directionality along ssDNA, although RepΔ2B monomers translocate with a slightly higher rate.

### Example 6

### Monomer STO Assays

DNA Unwinding Experiments. Stopped-flow experiments were performed using an Applied Photophysics SX18MV instrument (Leatherhead, U.K.), as described (Cheng et al., (2001) J. Mol. Biol. 310:327-50). Reactions were initiated by mixing a RepΔ2B-DNA solution with Buffer M (plus the indicated [NaCl]) containing 3 mM ATP, 4.2 mM MgCl₂, and 12 μM HP10T40 (concentration before mixing). STO quenched-flow DNA unwinding experiments were performed essentially as described (Cheng et al., (2001) J. Mol. Biol. 310:327-50; Ali and Lohman (1997) Science 275:377-80) by using an RQF-3 quenched-flow instrument (KinTek Instruments, University Park, Pa.). DNA II (FIG. 8 and Table 1) was labeled at the 5′ end with ³²P as described (Ali and Lohman (1997) Science 275:377-80). Reactions were initiated by mixing preformed RepΔ2B-DNA complex with Buffer M (plus the indicated [NaCl]) containing 3 mM ATP, 4.2 mM MgCl2, 12 μM HP10T40, and 10 μM unlabeled “top” strand of DNA II (Lucius et al., (2003) Biophys. J. 85:2224-39). HP10T40 is a DNA that possesses a 3′-(dT40) tail and a 10-bp hairpin and serves as a trap for free protein (Cheng et al., (2002) PNAS 99:16006-11), which ensures that DNA unwinding time courses are STO and thus that any DNA unwinding results exclusively from RepΔ2B that is prebound to the DNA. The unlabeled “top strand” serves as a DNA trap to prevent reannealing of released unwound strands. Control experiments showed that DNA unwinding was ATP-dependent, and RepΔ2B does not unwind a blunt-ended DNA under these conditions (data not shown). DNA unwinding time courses were analyzed by using Eq. 5 (Lucius et al., (2003) Biophys. J. 85:2224-39; Lucius et al., (2002) J. Mol. Biol. 324:409-28), based on Scheme 1:

$${f}_{ss} ( t ) = A ( ( 1 - \frac{Γ ( n , ( {k}_{obs} ) t )}{Γ ( n )} ) - {ⅇ}^{- {k}_{np} t} ( 1 - x ) {( \frac{{k}_{obs}}{{k}_{obs} - {k}_{NP}} )}^{n} ( 1 - \frac{Γ ( n , ( {k}_{obs} - {k}_{NP} ) t )}{Γ ( n )} ) ) \qquad Eq . 5$$

where fₛₛ(t) is the fraction of DNA molecules unwound at time, t; A is the total unwinding amplitude, including both phases of the reaction; n is the number of steps; k_{obs}=(k_{U}+k_{d}); and (1-x) is the fraction of DNA bound by helicase in nonproductive complexes, which subsequently isomerize with rate constant k_{NP} to form productive complexes. Γ(n) is the gamma function of n, and Γ(n, k_{obs} t), Γ(n, (k_{obs}-k_{Np})t) are the incomplete gamma functions of their arguments, respectively. In our analyses, we determine only k_{obs}, because we have insufficient information to determine k_{d}.

We performed a series of STO DNA unwinding kinetics experiments to compare the DNA helicase activity of RepΔ2B monomers vs. wtRep monomers. DNA unwinding was first monitored by using a fluorescently labeled DNA substrate possessing a 3′-(dT20) tail and an 18-bp duplex, with Cy3 and Cy5 fluorophores covalently attached to each strand at the blunt end of the duplex (DNA III; FIG. 8; Table 1). This DNA substrate allows the DNA unwinding time course to be followed in real time in a stopped-flow experiment by monitoring the changes in fluorescence resonance energy transfer (FRET) between the donor fluorophore, Cy3, and the acceptor fluorophore, Cy5 (Cheng et al., (2001) J. Mol. Biol. 310:327-50; Ha et al., (2002) Nature 419:638-641; Lucius et al., (2004) J. Mol. Biol. 339:731-50). A loss of FRET accompanies DNA unwinding and strand separation resulting in an increase in Cy3 fluorescence and a concomitant decrease in Cy5 fluorescence.

In the STO DNA unwinding kinetics experiments, RepΔ2B (80 nM) and DNA III (274 nM) were premixed in one syringe, in Buffer M plus 200 mM NaCl (25° C.) under conditions of a molar excess of DNA such that no more than one RepΔ2B monomer is bound per DNA. Reactions were initiated by mixing the RepΔ2B-DNA complex with a solution containing MgCl₂, ATP, and trap for free protein in Buffer M plus 200 mM NaCl. Such an experiment performed with the RepΔ2B monomer displayed a short lag phase followed by an increase in Cy3 fluorescence and a concomitant decrease in Cy5 fluorescence indicating DNA unwinding (FIG. 9B). The observation of lag kinetics in such an “all or none” unwinding assay reflects the fact that the helicase proceeds through multiple intermediate steps, each with similar rate constants, before the DNA is unwound completely, as shown in Scheme 1 (Ali et al., (1997) Science 275:377-80). NLLS fits of the Cy3 time course to Scheme 1 (Eq. 5) yields k_{obs}=(k_{U}+k_{d})=50.9 (±1.5) s⁻¹. In this analysis, we assumed a kinetic step size, m=L/n=4 bp, where L is the length of the duplex DNA in base pairs, and n is the number of steps in the kinetic scheme.

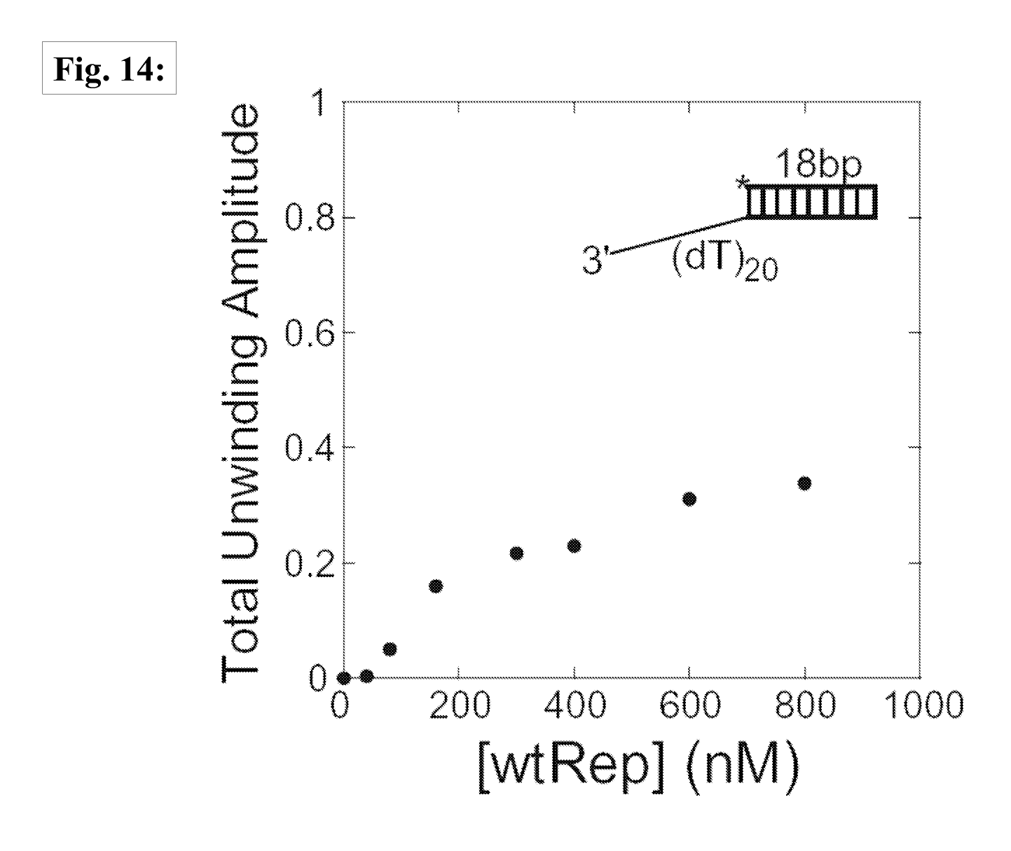

In contrast, FIG. 9B also shows that no DNA unwinding is detected in an identical experiment performed with wtRep protein when no more than one wtRep monomer is bound per DNA (200 nM wtRep preincubated with 600 nM DNA III). Therefore, a wtRep monomer is unable to unwind even this 18-bp DNA duplex. However, when the molar ratio of wtRep/DNA is increased to >2, then DNA unwinding is observed (FIG. 14) consistent with the need for dimerization of wtRep to activate its helicase activity (Cheng et al., (2001) J. Mol. Biol. 310:327-50).

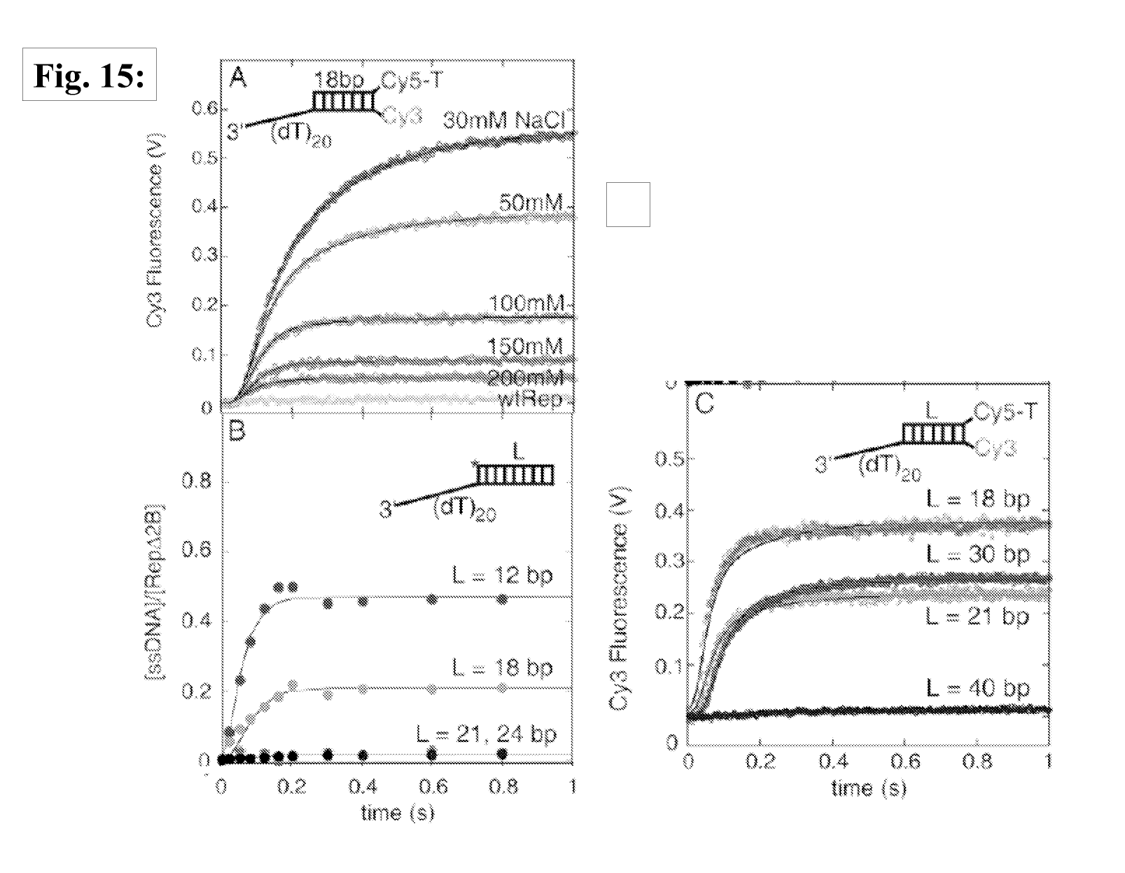

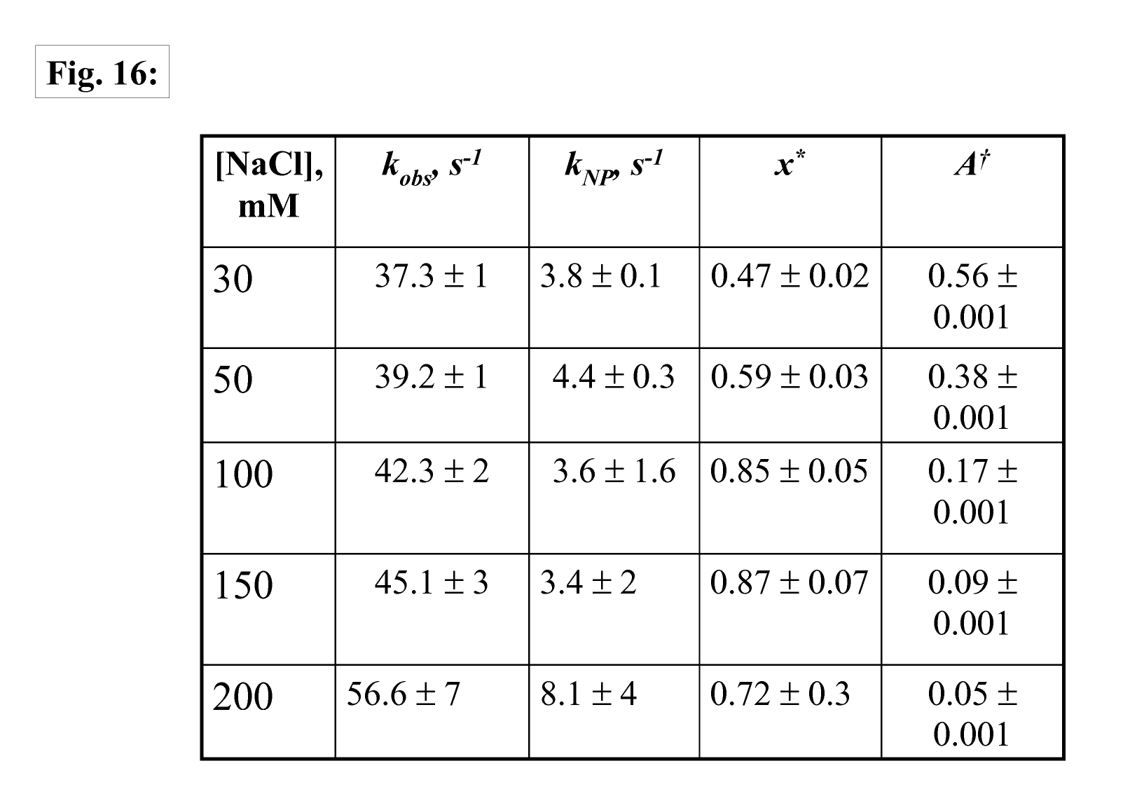

We also examined the effect of [NaCl] on DNA unwinding by the RepΔ2B monomer (FIG. 15A). STO DNA unwinding experiments were performed with 80 nM RepΔ2B and 320 nM DNA III in the stopped-flow, and DNA unwinding was monitored as changes in Cy3 fluorescence signal. FIG. 4A shows that the amplitude of DNA unwinding increases as [NaCl] is decreased. NLLS fits of these time courses to Scheme 1 (Eq. 1; FIG. 16) show that k_{obs}=(k_{U}+kd) increases with increasing [NaCl] from 37.3 (±1) s⁻¹ at 30 mM NaCl to 56.6 (±7) s⁻¹ at 200 mM NaCl (assuming a step size, m=4 bp). Although we are unable to obtain separate estimates for k_{U} and k_{d} from these fits, the increase in k_{obs} with increasing [NaCl] may be due partly to an increase in the dissociation rate constant, k_{d}, with increasing [NaCl]. These results suggest that the processivity of RepΔ2B monomer increases with decreasing [NaCl]. Identical experiments performed with wtRep showed no detectable DNA unwinding by wtRep monomer at any [NaCl] from 10 to 200 mM. STO DNA unwinding experiments were performed by using a series of DNA substrates with increasing duplex lengths, all possessing a 3′-(dT20) tail. Rapid chemical quenched flow experiments similar to those described (Ali and Lohman (1997) Science 275:377-80; Maluf et al., (2003) J. Mol. Biol. 325:913-35) were performed (Buffer M plus 200 mM NaCl, 25° C.). RepΔ2B (80 nM) was preincubated with DNA II (320 nM) radiolabeled with ³²P on the 5′ end of the top strand. FIG. 15B shows that the unwinding amplitude drops from ˜50% for a DNA substrate with a 12-bp duplex to <2% for DNA substrates with duplex lengths of 21 or 24 bp. A global NLLS fit of the data to Scheme 1 (Eq. 5), assuming m=4 bp, yields k_{obs}=(k_{U}+k_{d})=31.5 (±13.8) s⁻¹ at 200 mM NaCl. Stopped-flow DNA unwinding experiments were also performed with RepΔ2B as a function of duplex DNA length at 50 mM NaCl. FIG. 15C shows that decreasing the [NaCl] from 200 to 50 mM increases the unwinding amplitude of all duplex lengths examined, suggesting an increase in unwinding processivity. RepΔ2B monomer is able to unwind a 30-bp duplex at 50 mM NaCl, although no detectable unwinding of a 40-bp duplex is observed (FIG. 15C).

### Example 6

### UvrD Variant Helicase

After growth of E. Coli transformed with a plasmid that encode UvrDΔ2B, all plasmids that were recovered had additional sequences deleted from the ORF of the gene encoding for UvrDΔ2B. One of these plasmids was sequenced and found to encode a uvrDΔ2B gene with an additional deletion of amino acids 188-219. In wild-type UvrD, position 220 is the conserved aspartic acid residue of motif 11 that is essential for ATP hydrolysis. The lethality of the UvrDΔ2B mutant suggests that removal of the 2B domain of UvrD makes a more active and unregulated helicase that is lethal in E. Coli.

TABLE 1
DNA Sequences

| | | | |
|---|---|---|---|
| DNA | L* | Sequence | SEQ ID NO |

| | | | |
|---|---|---|---|
| I | 18 | 5′-d(GCCTCGCTGCCGTCGCCA)-3′ | SEQ ID NO:10 |
| | | 5′-d(Cy3 TGGCGACGGCAGCGAGGC | SEQ ID NO:11 |
| | | T₂₀)-3′ | |
| II | 12 | 5′-d(GCCTCGCTGCCG)-3′ | SEQ ID NO:12 |
| | | 5′-d(CGGCAGCGAGGCT₂₀)-3′ | SEQ ID NO:13 |
| | 18 | 5′-d(GCCTCGCTGCCGTCGCCA)-3′ | SEQ ID NO:14 |
| | | 5′-d(TGGCGACGGCAGCGAGGCT₂₀)-3′ | SEQ ID NO:15 |
| | 21 | 5′-d(GCCTCGCTGCCGTCGCCAGTC)-3′ | SEQ ID NO:16 |
| | | 5′-d(GACTGGCGACGGCAGCGAGGCT₂₀)-3′ | SEQ ID NO:17 |
| | 24 | 5′-d(GCCCTGCTGCCGAACAACGAAGGT)- | SEQ ID NO:18 |
| | | 3′ | |
| | | 5′-d(ACCTTCGTTGGTCGGCAGCAGGGC | SEQ ID NO:19 |
| | | T₂₀)-3′ | |
| III | 18 | 5′-d(GCCTCGCTGCCGTCGCCACy5-T)- | SEQ ID NO:20 |
| | | 3′ | |
| | | 5′-d(Cy3 TGGCGACGGCAGCGAGGC | SEQ ID NO:21 |
| | | T₂₀)-3′ | |
| | 21 | 5′-d(GCCCTGCTGCCGACCAACGAA Cy5- | SEQ ID NO:22 |
| | | T)-3′ | |
| | | 5′-d(Cy3 TTCGTTGGTCGGCAGCAGGGC | SEQ ID NO:23 |
| | | T₂₀)-3′ | |
| | 30 | 5′-d(CGACCAACGATGGTTACATTCCCGCT | SEQ ID NO:24 |
| | | GGTG Cy5-T)-3′ | |
| | | 5′-d(Cy3 CAGCAGCGGGAATGTAACCATC | SEQ ID NO:25 |
| | | GTTGGTCGT₂₀)-3′ | |
| | 40 | 5′-d(ACCCTGCTGCCGACCAACGATGGTTA | SEQ ID NO:26 |
| | | CATTCCCGCT-GCTG Cy5-T)-3′ | |
| | | 5′-d(Cy3 CAGCAGCGGGAATGTAACCATC | SEQ ID NO:27 |
| | | GTTGGTCGGCA-GCAGGGCT₂₀)-3′ | |

*Length of duplex region of DNA in base pairs.

What is claimed:

1. An in vitro method of unwinding a double helix, the method comprising contacting the double helix with a variant helicase that lacks structural autoinhibition of helicase activity.

2. The method of claim 1, wherein the variant helicase is a monomer.

3. The method of claim 2, wherein the helicase is a variant of Rep.

4. The method of claim 3, wherein the variant helicase has a sequence comprising the amino acid sequence of SEQ ID NO:1 or the nucleic acid sequence of SEQ ID NO:2.

5. The method of claim 3, wherein the variant helicase is comprised of a sequence with at least 95% sequence identity to SEQ ID NO:1 or SEQ ID NO:2 and lacks structural autoinhibition of helicase activity.

6. The method of claim 1, wherein the double helix is unwound at a temperature between about 20° C. and about 30° C.

7. The method of claim 1, further comprising replicating the unwound double helix.

8. An in vitro composition comprising a variant helicase that lacks structural autoinhibition of helicase activity and a polymerase.

9. The composition of claim 9, wherein the variant helicase is a monomer.

10. The composition of claim 10, wherein the helicase is a variant of Rep.

11. The composition of claim 11, wherein the variant helicase has a sequence comprising the amino acid sequence of SEQ ID NO:1 or the nucleic acid sequence of SEQ ID NO:2.

12. The composition of claim 11, wherein the variant helicase is comprised of a sequence with at least 95% sequence identity to SEQ ID NO:1 or SEQ ID NO:2 and lacks structural autoinhibition of helicase activity.

13. The composition of claim 9, further comprising a double-stranded nucleic acid helix.

14. A kit comprising a variant helicase that lacks structural autoinhibition of helicase activity, and a polymerase.

15. The kit of claim 16, wherein the variant helicase is a monomer.

16. The kit of claim 17, wherein the helicase is a variant of Rep.

17. The kit of claim 17, wherein the variant helicase has a sequence comprising the amino acid sequence of SEQ ID NO:1 or the nucleic acid sequence of SEQ ID NO:2.

18. The kit of claim 11, wherein the variant helicase is comprised of a sequence with at least 95% sequence identity to SEQ ID NO:1 or SEQ ID NO:2 and lacks structural autoinhibition of helicase activity.

19. The kit of claim 16, wherein the kit is used for a procedure selected from the group consisting of PCR, isothermal PCR, replication of genomic DNA, isothermal replication of genomic DNA, in vitro transcription, in vitro translation, and reverse transcription.

20. The kit of claim 22, wherein the procedure is performed at a temperature between about 20° C. and about 30° C.


SEQUENCE LISTING (27 sequences)

SEQ ID NO: 1 - protein, 508 aa, Escherichia coli
MRLNPGQQQAVEFVTGPCLVLAGAGSGKTRVITNKIAHLIRGCGYQARHIAAVTFTNKAAREMKERVGQTLGRKEARGLMISTFHTLGLDIIKREYAALGMKANFSLFDDTDQLALLKELTEGLIEDDKVLLQQLISTISNWKNDLKTPSQAAASAIGERDRIFAHCYGLYDAHLKACNVLDFDDLILLPTLLLQRNEEVRKRWQNKIRYLLVDEYQDTNTSQYELVKLLVGSRARFTVVGDDDQSIYSWRGARPQNLVLLSQDFPALKVIKLEQNYRSSGRILKAANILIANNPHVFEKRLFSELGYGAELKVLSANNEEHEAERVTGELIAHHFVNKTQYKDYAILYRGNHQSRVFEKFLMQNRIPYKISGGGGGGESEEELDQVQLMTLHASKGLEFPYVYMVGMEEGFLPHQSSIDEDNIDEERRLAYVGITRAQKELTFTLCKERRQYGELVRPEPSRFLLELPQDDLIWEQERKVVSAEERMQKGQSHLANLKAMMAAKRGK

SEQ ID NO: 2 - DNA, 1524 bp, Escherichia coli
atgcgtctaaaccccggccaacaacaagctgtcgaattcgttaccggcccctgcctggtgctggcgggcgcgggttccggtaaaactcgtgttatcaccaataaaatcgcccatctgatccgcggttgcggttatcaggcgcggcacattgcggcggtgacctttactaataaagcagcgcgcgagatgaaagagcgtgtagggcagacgctggggcgcaaagaggcgcgtgggctgatgatctccactttccatacgttggggctggatatcatcaaacgcgagtatgcggcgcttgggatgaaagcgaacttctcgttgtttgacgataccgatcagcttgctttgcttaaagagttgaccgaggggctgattgaagatgacaaagttctcctgcaacaactgatttcgaccatctctaactggaagaatgatctcaaaacaccgtcccaggcggcagcaagtgcgattggcgagcgggaccgtatttttgcccattgttatgggctgtatgatgcacacctgaaagcctgtaacgttctcgacttcgatgatctgattttattgccgacgttgctgctgcaacgcaatgaagaagtccgcaagcgctggcagaacaaaattcgctatctgctggtggatgagtatcaggacaccaacaccagccagtatgagctggtgaaactgctggtgggcagccgcgcgcgctttaccgtggtgggtgacgatgaccagtcgatctactcctggcgcggtgcacgtccgcaaaacctggtgctgctgagtcaggattttccggcgctgaaggtgattaagcttgagcagaactatcgctcttccgggcgtattctgaaagcggcgaacatcctgatcgccaataacccgcacgtctttgaaaagcgtctgttctccgaactgggttatggcgcggagctaaaagtattaagcgcgaataacgaagaacatgaggctgagcgcgttactggcgagctgatcgcccatcacttcgtcaataaaacgcagtacaaagattacgccattctttatcgcggtaaccatcagtcgcgggtgtttgaaaaattcctgatgcaaaaccgcatcccgtacaaaatatctggcggtggcggtggcggtgagagtgaagaagagctggatcaggtgcaactgatgactctccacgcgtcgaaagggctggagtttccttatgtctacatggtcggtatggaagaagggtttttgccgcaccagagcagcatcgatgaagataatatcgatgaggagcggcggctggcctatgtcggcattacccgcgcccagaaggaattgacctttacgctgtgtaaagaacgccgtcagtacggcgaactggtgcgcccggagccgagccgctttttgctggagctgccgcaggatgatctgatttgggaacaggagcgcaaagtggtcagcgccgaagaacggatgcagaaagggcaaagccatctggcgaatctgaaagcgatgatggcggcaaaacgagggaaa

SEQ ID NO: 3 - DNA, 30 bp, Escherichia coli
atcttttccggctcgagttatttccctcgt

SEQ ID NO: 4 - DNA, 30 bp, Escherichia coli
accgccaccgccaccgccagatattttgta

SEQ ID NO: 5 - DNA, 30 bp, Escherichia coli
cggtggcggtggcggtgagagtgaagaaga

SEQ ID NO: 6 - DNA, 34 bp, Escherichia coli
gagcaatacacatatgcgtctaaaccccggccaa

SEQ ID NO: 7 - DNA, 39 bp, Escherichia coli
gaccagtcgatctactccgctcgcggtgcacgtccgcaa

SEQ ID NO: 8 - protein, 20 aa, Escherichia coli
MGSSHHHHHHSSGLVPRGSH

SEQ ID NO: 9 - DNA, 18 bp, Artificial
  completely artificial
gcctcgctgccgtcgcca

SEQ ID NO: 10 - DNA, 18 bp, Artificial
  E. COLI
gcctcgctgccgtcgcca

SEQ ID NO: 11 - DNA, 19 bp, Artificial
  E. COLI
  misc_feature (1)..(1): Cy3 tag
  misc_feature (19)..(19): 20 repeats
tggcgacggcagcgaggct

SEQ ID NO: 12 - DNA, 12 bp, Artificial
  E. coli
gcctcgctgccg

SEQ ID NO: 13 - DNA, 13 bp, Artificial
  E. coli
  misc_feature (13)..(13): 20 repeats
cggcagcgaggct

SEQ ID NO: 14 - DNA, 18 bp, Artificial
  E. coli
gcctcgctgccgtcgcca

SEQ ID NO: 15 - DNA, 19 bp, Artificial
  E. coli
  misc_feature (19)..(19): 20 repeats
tggcgacggcagcgaggct

SEQ ID NO: 16 - DNA, 21 bp, Artificial
  E. coli
gcctcgctgccgtcgccagtc

SEQ ID NO: 17 - DNA, 22 bp, Artificial
  E. coli
  misc_feature (22)..(22): 20 repeats
gactggcgacggcagcgaggct

SEQ ID NO: 18 - DNA, 24 bp, Artificial
  E. coli
gccctgctgccgaacaacgaaggt

SEQ ID NO: 19 - DNA, 25 bp, Artificial
  E. coli
  misc_feature (25)..(25): 20 repeats
accttcgttggtcggcagcagggct

SEQ ID NO: 20 - DNA, 19 bp, Artificial
  E. coli
  misc_feature (19)..(19): Cy5 label
gcctcgctgccgtcgccat

SEQ ID NO: 21 - DNA, 19 bp, Artificial
  E. coli
  misc_feature (1)..(1): Cy3 label
  misc_feature (19)..(19): 20 repeats
tggcgacggcagcgaggct

SEQ ID NO: 22 - DNA, 22 bp, Artificial
  E. coli
  misc_feature (22)..(22): Cy5 label
gccctgctgccgaccaacgaat

SEQ ID NO: 23 - DNA, 22 bp, Artificial
  E. coli
  misc_feature (1)..(1): Cy3 label
  misc_feature (22)..(22): 20 repeats
ttcgttggtcggcagcagggct

SEQ ID NO: 24 - DNA, 31 bp, Artificial
  E. coli
  misc_feature (31)..(31): Cy5 label
cgaccaacgatggttacattcccgctggtgt

SEQ ID NO: 25 - DNA, 31 bp, Artificial
  E. coli
  misc_feature (1)..(1): Cy3 label
  misc_feature (31)..(31): 20 repeats
cagcagcgggaatgtaaccatcgttggtcgt

SEQ ID NO: 26 - DNA, 41 bp, Artificial
  E. coli
  misc_feature (41)..(41): Cy5 label
accctgctgccgaccaacgatggttacattcccgctgctgt

SEQ ID NO: 27 - DNA, 41 bp, Artificial
  E. coli
  misc_feature (1)..(1): Cy3 label
  misc_feature (41)..(41): 20 repeats
cagcagcgggaatgtaaccatcgttggtcggcagcagggct